(19) United States
(12) Patent Application Publication
(10) Pub. No.: US 2013/0055472 A1
(43) Pub. Date: Feb. 28, 2013

(54) Methods for tissue culture and transformation of sugarcane

(71) Applicants: Myeong-Je Cho, Santa Clara, CA (US); Theodore M. Klein, Wilmington, DE (US); Zuo-Yu Zhao, Johnston, IA (US)

(72) Inventors: Myeong-Je Cho, Santa Clara, CA (US); Theodore M. Klein, Wilmington, DE (US); Zuo-Yu Zhao, Johnston, IA (US)

(73) Assignees: E.I. du Pont de Nemours and Company, Wilmington, DE (US); Pioneer Hi Bred International Inc., Johnston, IA (US)

(21) Appl. No.: 13/599,003

(22) Filed: Aug. 30, 2012

Related U.S. Application Data
(60) Provisional application No. 61/529,593, filed on Aug. 31, 2011.

(51) Int. Cl. A01H 1/06; C12N 5/04

(52) U.S. Cl. 800/294; 435/430; 800/278

(57) ABSTRACT

Compositions and methods for the efficient transformation and regeneration of monocot plants are provided. The methods of transformation involve infection with Agrobacterium. In this manner, any gene of interest can be introduced into the monocot plant with high transformation efficiency and in low copy number. Transformed and regenerated monocot cells, tissues, plants, and seed are also provided. The invention encompasses regenerating transformed plants, transgenic seeds produced therefrom, and transgenic plants and transgenic seeds from subsequent generations.

### CROSS-REFERENCE TO RELATED APPLICATIONS

This application claims the benefit of U.S. Provisional Patent Application No. 61/529,593, filed Aug. 31, 2011, which is hereby incorporated herein in its entirety by reference.

### REFERENCE TO SEQUENCE LISTING SUBMITTED ELECTRONICALLY

The official copy of the sequence listing is submitted electronically via EFS-Web as an ASCII formatted sequence listing with a file named “3676_sequence_listing_{—}20120831.TXT” created on Aug. 17, 2012, and having a size of 32 kilobytes and is filed concurrently with the specification. The sequence listing contained in this ASCII formatted document is part of the specification and is herein incorporated by reference in its entirety.

### FIELD OF THE INVENTION

The present disclosure relates to the field of plant biotechnology. More particularly, the present invention relates to methods for culturing and transforming monocotyledonous plants such as, for example, sugarcane.

### BACKGROUND OF THE INVENTION

Cultivated monocotyledonous crops such as corn and sugarcane have substantial commercial value throughout the world. The development of scientific methods useful in improving the quantity and quality of sugarcane and other crops is, therefore, of significant commercial interest. Significant effort has been expended to improve the quality of cultivated monocotyledonous crop species by conventional plant breeding. Methods of conventional plant breeding have been limited, however, to the movement of genes and traits between plant varieties.

In addition to traditional breeding techniques, incorporation of disease resistance, increased or modified oil content, and other desirable traits can be envisioned using the modern tools of molecular biology including plant genetic engineering. Plant genetic engineering involves the transfer of a desired gene or genes into the inheritable germline of crop plants such that those genes can be bred into or among the elite varieties used in modern agriculture. Gene transfer techniques allow the development of new classes of crop varieties with improved disease resistance, herbicide tolerance, and increased nutritional value.

Most current genetic engineering technologies require that genes be delivered to cells grown in vitro. For example, most published methods for generating transformed plants from cereals (e.g., rice, wheat, maize, oat, sorghum, triticale, barley and rye) utilize as initial explants the immature scutellum of the embryo or microspores directly or tissue derived from immature embryos or microspores. From these initial explants, cellular proliferation occurs. After selection or screening for transformants, plants are regenerated.

Aspects of in vitro culturing and/or transformation process are likely to be responsible for problems encountered in efforts to genetically engineer plant species, including monocots such as cereals and grasses. Most transformation protocols require that the target tissue undergo embryogenesis, which may include de-differentiation of a single original transformed cell before the sustained cell divisions that give rise to an embryo consisting mostly or entirely of cells that contain the introduced DNA. De-differentiation during in vitro culturing introduces stresses on the genome, causing modifications of the genome that are associated with somaclonal variation, including DNA methylation, point mutations, deletions, insertions, and the generation of gross cytogenetic abnormalities. These genomic modifications lead to subsequent phenotypic abnormalities and performance losses and may contribute to the other problems listed above. Other transformation target tissues are organogenic tissues such as green regenerative tissue and shoot meristematic cultures. Transformation of these tissues causes fewer somaclonal and phenotypic variations.

Accordingly, there remains a need for improved transformation and regeneration methods to promote the engineering of desirable traits into agronomically important crops. In addition, there remains a need for highly efficient transformation methods that yield regenerable plant tissue.

### SUMMARY OF THE INVENTION

Methods and compositions are provided for the efficient regeneration of a monocotyledonous plant. More particularly, methods and compositions of the present invention find use in agriculture for the transformation and regeneration of a monocotyledonous plant such as, for example, sugarcane. The present invention involves methods for inducing callus and green regenerative tissue formation from in vitro-cultured buds or in vitro-cultured whole plantlets. Transformation of green regenerative tissue cultures results in long-term regenerability and less somaclonal variation in monocot crops, as compared with standard callus systems. Accordingly, the methods provided herein establish an efficient and less genotype-specific tissue culture and transformation system for regenerating monocot plants such as sugarcane. The methods further involve media comprising benzylaminopurine (BAP), 2,4-dichlorophenoxyacetic acid (2,4-D), and/or copper. In this manner, the invention provides methods for the production of callus and green regenerative tissue that is highly amenable to regeneration and transformation from cultivars of monocots such as sugarcane that are otherwise recalcitrant to transformation. The present invention also provides methods directed to Agrobacterium-mediated transformation of callus or green regenerative plant tissue with a gene of interest. In this manner, any gene of interest can be introduced into a fertile or infertile plant with high transformation efficiency. The transferred gene will be present in the transformed plant in low copy number. Expression of a coding sequence by such a transformed plant will result in the production of a polypeptide of interest in the transformed plant.

The following embodiments are encompassed by the present invention.
1. A method of regenerating a plant, the method comprising the steps of:

(a) culturing an in vitro-cultured plantlet or in vitro-cultured bud in the presence of a medium to induce callus or green regenerative tissue formation;

(b) culturing the callus or green regenerative tissue in the presence of a medium to regenerate the plant.
2. The method according to embodiment 1, wherein the plantlet is an in vitro-cultured whole plantlet.
3. The method according to any one or more of embodiments 1 to 2, wherein roots of the plantlet are removed prior to culturing step (a).
4. The method according to any one or more of embodiments 1 to 3, wherein the plantlet or in vitro cultured bud is monocotyledonous.
5. The method according to any one or more of embodiments 1 to 4, wherein the monocotyledonous plantlet or in vitro cultured bud is sugarcane.
6. The method according to any one or more of embodiments 1 to 5, further comprising introducing a nucleic acid into a cell of the callus or green regenerative tissue to produce a transformed plant cell.
7. The method according to any one or more of embodiments 1 to 6, wherein the medium of (a) comprises benzylaminopurine (BAP), 2,4-dichlorophenoxyacetic acid (2,4-D), and copper, and further comprises at least one of maltose, thiamine-HCl, myo-inositol, N—Z-amine-A (casein hydrolysate), and proline.
8. The method according to any one or more of embodiments 1 to 7, wherein the medium of (a) comprises about 0.01 to about 5 milligrams/Liter BAP, about 0.1 to about 5 milligrams/Liter 2,4-D, about 0.1 to about 50 μM copper, about 0.25 grams/Liter myo-inositol, about 1 gram/Liter N—Z-amine-A (casein hydrolysate), and about 0.7 grams/Liter proline.
9. The method according to any one or more of embodiments 1 to 8, wherein the medium of (a) comprises about 0.5 to about 1.0 milligrams/L BAP, about 1 gram/L 2,4-D, about 30 grams/L maltose, about 0.2 milligrams/L thiamine-HCl, about 4.9 μM copper, about 0.25 grams/L myo-inositol, about 1 gram/L N—Z-amine-A (casein hydrolysate), and about 0.7 grams/L proline.
10. The method according to any one or more of embodiments 1 to 9, wherein the medium of (a) comprises benzylaminopurine (BAP), and 2,4-dichlorophenoxyacetic acid (2,4-D), and further comprises at least one of maltose, thiamine-HCl, myo-inositol, N—Z-amine-A (casein hydrolysate), and proline.
11. The method according to any one or more of embodiments 1 to 10, wherein the medium of (a) comprises about 0.01 to about 5 milligrams/Liter BAP, about 0.1 to about 5 milligrams/Liter 2,4-D, about 0.25 grams/Liter myo-inositol, about 1 gram/Liter N—Z-amine-A (casein hydrolysate), and about 0.7 grams/Liter proline.
12. The method according to any one or more of embodiments 1 to 11, wherein the medium of (a) comprises about 0.5 to about 1.0 milligrams/L BAP, about 1 gram/L 2,4-D, about 30 grams/L maltose, about 0.2 milligrams/L thiamine-HCl, about 0.25 grams/L myo-inositol, about 1 gram/L N—Z-amine-A (casein hydrolysate), and about 0.7 grams/L proline.
13. A method for producing a transformed plant, comprising the steps of:
(a) culturing a in vitro-cultured plantlet or in vitro-cultured bud in the presence of an medium to induce callus or green regenerative tissue formation;
(b) introducing a nucleic acid into a cell of the callus or green regenerative tissue to produce a transformed plant cell,
(c) culturing the transformed plant cell in the presence of a medium, thereby promoting proliferation and formation of a transformed structure that is competent to regenerate; and
(d) culturing the transformed structure in the presence of a medium to produce the transformed plant.
14. The method according to embodiment 13, wherein the plantlet is an in vitro-cultured whole plantlet.
15. The method according to any one or more of embodiments 13 to 14, wherein roots of the plantlet are removed prior to culturing step (a).
16. The method according to any one or more of embodiments 13 to 15, wherein the introducing step comprises Agrobacterium-mediated transformation.
17. The method according to any one or more of embodiments 13 to 16, wherein the introducing step comprises bombardment of the callus or green regenerative tissue with microprojectiles coated with the nucleic acid.
18. The method according to any one or more of embodiments 13 to 17, wherein the medium of (a) comprises benzylaminopurine (BAP), 2,4-dichlorophenoxyacetic acid (2,4-D), and copper, and further comprises at least one of maltose, thiamine-HCl, myo-inositol, N—Z-amine-A (casein hydrolysate), and proline.
19. The method according to any one or more of embodiments 13 to 18, wherein the medium of (a) comprises about 0.01 to about 5 milligrams/Liter BAP, about 0.1 to about 5 milligrams/Liter 2,4-D, about 0.1 to about 50 μM copper, about 0.25 grams/Liter myo-inositol, about 1 gram/Liter N—Z-amine-A (casein hydrolysate), and about 0.7 grams/Liter proline.
20. The method according to any one or more of embodiments 13 to 19, wherein the medium of (a) comprises about 0.5 to about 1.0 milligrams/L BAP, about 1 gram/L 2,4-D, about 30 grams/L maltose, about 0.2 milligrams/L thiamine-HCl, about 4.9 μM copper, about 0.25 grams/L myo-inositol, about 1 gram/L N—Z-amine-A (casein hydrolysate), and about 0.7 grams/L proline.
21. The method according to any one or more of embodiments 13 to 20, wherein the medium of (a) comprises benzylaminopurine (BAP), 2,4-dichlorophenoxyacetic acid (2,4-D), and further comprises at least one of maltose, thiamine-HCl, myo-inositol, N—Z-amine-A (casein hydrolysate), and proline.
22. The method according to any one or more of embodiments 13 to 21, wherein the medium of (a) comprises about 0.01 to about 5 milligrams/Liter BAP, about 0.1 to about 5 milligrams/Liter 2,4-D, about 0.25 grams/Liter myo-inositol, about 1 gram/Liter N—Z-amine-A (casein hydrolysate), and about 0.7 grams/Liter proline.
23. The method according to any one or more of embodiments 13 to 22, wherein the medium of (a) comprises about 0.5 to about 1.0 milligrams/L BAP, about 1 gram/L 2,4-D, about 30 grams/L maltose, about 0.2 milligrams/L thiamine-HCl, about 0.25 grams/L myo-inositol, about 1 gram/L N—Z-amine-A (casein hydrolysate), and about 0.7 grams/L proline.
24. The method according to any one or more of embodiments 13 to 23, further comprising selecting for the transformed plant cell by incubating the callus or green regenerative tissue in the presence of a medium comprising a selective agent.
25. The method according to any one or more of embodiments 13 to 24, wherein the plant, in vitro-cultured bud, or plantlet is monocotyledonous.
26. The method according to any one or more of embodiments 13 to 25, wherein the monocotyledonous plant, bud culture, or plantlet is sugarcane.
27. A method for producing a transformed plant, comprising the steps of:

(a) culturing a whole plantlet or in vitro-cultured bud in the presence of a medium to induce callus or green regenerative tissue formation;

(b) contacting the callus or green regenerative tissue with an Agrobacterium comprising a vector which comprises a polynucleotide, wherein the polynucleotide comprises an expression cassette comprising a gene which confers resistance to a selection agent;

(c) co-cultivating the tissue with the Agrobacterium;

(d) selecting regenerable cells comprising the polynucleotide; and

(e) culturing the regenerable cells in the presence of a regeneration medium to produce the transformed plant.
28. The method according to embodiment 27, further comprising culturing the tissue of step (c) in a medium comprising compound capable of inhibiting the growth of Agrobacterium and the selection agent.
29. The method according to any one or more of embodiments 27 to 28, wherein the plantlet is an in vitro-cultured whole plantlet.
30. The method according to any one or more of embodiments 27 to 29, wherein roots of the plantlet are removed prior to culturing step (a).
31. The method according to any one or more of embodiments 27 to 30, wherein the medium of (a) comprises benzylaminopurine (BAP), 2,4-dichlorophenoxyacetic acid (2,4-D), and copper, and further comprises at least one of maltose, thiamine-HCl, myo-inositol, N—Z-amine-A (casein hydrolysate), and proline.
32. The method according to any one or more of embodiments 27 to 31, wherein the medium of (a) comprises about 0.01 to about 5 milligrams/Liter BAP, about 0.1 to about 5 milligrams/Liter 2,4-D, about 1.0 to about 50 μM copper, about 0.25 grams/Liter myo-inositol, about 1 gram/Liter N—Z-amine-A (casein hydrolysate), and about 0.7 grams/Liter proline.
33. The method according to any one or more of embodiments 27 to 32, wherein the medium of (a) comprises benzylaminopurine (BAP), 2,4-dichlorophenoxyacetic acid (2,4-D), and further comprises at least one of maltose, thiamine-HCl, myo-inositol, N—Z-amine-A (casein hydrolysate), and proline.
34. The method according to any one or more of embodiments 27 to 33, wherein the medium of (a) comprises about 0.01 to about 5 milligrams/Liter BAP, about 0.1 to about 5 milligrams/Liter 2,4-D, about 0.25 grams/Liter myo-inositol, about 1 gram/Liter N—Z-amine-A (casein hydrolysate), and about 0.7 grams/Liter proline.
35. The method according to any one or more of embodiments 27 to 34, wherein the medium of (c) comprises about 4.43 grams/L MS salts and vitamins, 20 grams/L sucrose, 1 gram/L myo-inositol and 3.5 grams/L Phytagel.
36. The method according to any one or more of embodiments 27 to 35, wherein the plant, plant bud, or plantlet is monocotyledonous.
37. The method according to any one or more of embodiments 27 to 36, wherein the monocotyledonous plant, plant bud, or plantlet is sugarcane.
38. The method according to any one or more of embodiments 27 to 37, wherein the polynucleotide further comprises a developmental gene cassette.
39. The method according to embodiment 38, wherein the developmental gene cassette encodes at least one cell proliferation transcription factor selected from the group consisting of either of babyboom (BBM) or Wuschel (WUS), or both.
40. The method according to any one or more of embodiments 38 to 39, wherein the developmental gene cassette comprises at least one nucleotide sequence selected from the group consisting of SEQ ID NOs:1 and 3.
41. The method according to any one or more of embodiments 38 to 40, wherein the developmental gene cassette comprises at least one nucleotide sequence selected from the group consisting of SEQ ID NOs:5, 7, 9, and 11.
42. The method according to any one or more of embodiments 38 to 40, wherein the developmental gene cassette comprises at least one nucleotide sequence selected from the group consisting of SEQ ID NOs:1 and 3 and at least one nucleotide sequence selected from the group consisting of SEQ ID NOs:5, 7, 9, and 11.
43. The method according to any one or more of embodiments 27 to 37 wherein the Agrobacterium is an auxotrophic strain.

Additional embodiments of the invention are described herein below.

### BRIEF DESCRIPTION OF THE DRAWINGS

FIG. 1 provides photographs depicting various stages in the induction of callus and green regenerative tissue from in vitro-cultured plantlet and in vitro-cultured buds.

FIG. 2 provides photographs depicting various stages in the induction of callus and green regenerative tissue from in vitro-cultured plantlet and in vitro-cultured buds.

FIG. 3 provides photographs depicting callus and green tissue induction from in vitro-cultured whole plantlets.

FIG. 4 provides photographs depicting various stages in the induction of green regenerative tissue.

FIG. 5 provides photographs depicting transgenic sugarcane events on bialaphos selection.

FIG. 6 provides photographs depicting plant regeneration from T₀ CP89-2376 events.

FIG. 7 provides a photograph depicting T₀ CP89-2376 plants in soil.

FIG. 8 provides images depicting transgenic CP89-2376 events showing fluorescent RFP expression and regenerability.

FIG. 9 provides images depicting transgenic CP89-2376 events showing fluorescent CFP expression and regenerability.

FIG. 10 provides images depicting transgenic CP89-2376 events showing fluorescent YFP expression.

FIG. 11 provides images depicting fluorescent DsRED and YFP expression in T₀ CP89-2376 events.

### DETAILED DESCRIPTION OF THE INVENTION

The invention is drawn to methods and compositions for regenerating and transforming monocotyledonous plants, particularly sugarcane. The methods of the present invention are directed to the transformation of a monocotyledonous plant with a gene of interest, including methods involving, for example, Agrobacterium-mediated transformation and microparticle bombardment. In this manner, any gene of interest can be introduced into a monocotyledonous plant with high transformation efficiency. The transferred gene will be present in the transformed plant in low copy number. Transformed plants, plant cells, and seeds are also disclosed herein. Compositions of the invention include media comprising high concentrations of benzylaminopurine (BAP), low concentrations of 2,4-dichlorophenoxyacetic acid (2,4-D), and/or copper. Compositions further comprise at least one of maltose, thiamine-HCl, myo-inositol, N—Z-amine-A (casein hydrolysate) from bovine milk, and proline.

In one aspect, methods for inducing callus formation are provided. For example, methods are provided for inducing callus formation from in vitro-cultured whole plantlets or in vitro-cultured buds derived from meristematic tissue. Such plant tissues can be cultured in the presence of a medium provided herein to induce callus and green regenerative tissue formation. In such cases, meristematic tissue can include, without limitation, plantlets and leaf whorls. As used herein, the term “callus” is intended to include regenerable plant tissue such as embryogenic callus. As used herein, “plantlet” includes reference to young or small plants used as propagules. Plantlets may be produced asexually by tissue culture or cell culture. As used herein, “in vitro-cultured bud” includes in vitro-propagated apical and axillary buds. Plant apical and axillary buds are small terminal or lateral protuberances on the stem of a vascular plant that may develop into a flower, leaf, or shoot. Plant buds arise from meristem tissue and may consist of overlapping immature leaves or petals. The use of such in vitro-cultured plant materials can be more convenient for handling without contamination than field-grown plant materials such as immature leaf whorls. For use in the methods provided herein, roots of the plantlet may be removed prior to culturing in the presence of an induction medium provided herein, but removal of the roots is not necessary.

Methods for producing in vitro-cultured whole plantlets and in vitro-cultured buds are described by Hendre et al. (1983) Sugar Cane 1:5-8, Cheema and Hussain (2004), Intl. J. Agriculture & Biol. 6(2): 257-259, Manickavasagam et al. (2004) Plant Cell Rep. 23:134-143, and Ali et al. (2008) Pak. J. Bot. 40(1): 139-149, each of which is herein incorporated by reference in its entirety. Briefly, apical portions consisting of mature apical and axillary buds of healthy stalks of sugarcane plants can be collected from the field or greenhouse. In some cases, apical and axillary buds containing meristem cells are isolated from the cane stalk, surface-sterilized, and inoculated. In vitro-cultured plantlets can be regenerated, proliferated, and maintained on medium with or without phytohormone.

In another aspect, methods for inducing green regenerative tissue formation are provided. As described herein, any meristematic tissue can be cultured in a medium provided herein to directly induce green regenerative tissue formation. As used herein, the term “green regenerative tissue” is intended to include green callus tissue, meristematic tissue, or other regenerative tissue suitable for transformation and/or regeneration into a plant. These tissues appear to require only a simple redirection of cells in the tissues for the formation of shoots and plants to occur in culture, unlike cells derived from an immature embryo or microspore which require an apparent de-differentiation process. Meristematic tissue can include, without limitation, plant tissue in in vitro cultured buds, micropropagated plantlets, and leaf whorls. For use in the methods provided herein, plantlets need not be cut or otherwise dissected. For example, whole plantlets comprising meristematic tissue may be cultured in an induction medium provided herein to induce green regenerative tissue suitable for transformation using Agrobacterium.

In a further aspect, methods for regenerating a monocotyledonous plant are provided. For example, callus and green regenerative tissues induced according to the methods provided herein find use in methods for regenerating transformed or non-transformed plants.

In some embodiments, methods for regenerating a plant can include the steps of:

(a) culturing a whole plantlet or in vitro-cultured buds in the presence of a medium to induce callus or green regenerative tissue formation; and

(b) culturing the callus or green regenerative tissue in the presence of a medium to regenerate the plant.

In a first culturing step, an in vitro-cultured whole plantlet or in vitro-cultured bud can be cultured in the presence of a medium to induce callus or green regenerative tissue formation. The medium can comprise BAP, 2,4-D, and/or copper. The medium may include copper or have additional copper from another source, for example, from MS salts. The concentration of copper in the medium may range from about 0.1 μM to about 50 μM. In certain embodiment, the medium comprises about 0.1 μM, 0.5 μM, 1 μM, 2 μM, 3 μM, 4 μM, 5 μM or greater concentration of copper. Any form of copper that provides nutritional source for plant tissue culture may be utilized including but not limited to salts or compositions containing copper e.g. cupric sulfate, copper chloride, copper nitrate, copper gluconate, or copper acetate. In some embodiments, a medium can comprise any well-known auxin or cytokinin in the practice of the invention. Auxins include, but are not limited to, 2,4-D, dicamba, picloram, IAA and 2,4,5-T, and NAA. Cytoknins include, but are not limited to, BAP, kinetin, zeatin, zeatin riboside, and N⁶-(2-isopentenyl) adenine (2iP). A particular genotype or species may respond optimally to a specific phytohormone, For example, about 0.01 mg/L to about 5.0 mg/L BAP, and about 0.1 mg/L to about 5.0 mg/L 2,4-D. In some embodiments, the BAP is present at about 0.5 milligrams/Liter and the 2,4-D is present at about 1 milligram/Liter.

Media provided herein may further comprise at least one of maltose, thiamine-HCl, myo-inositol, N—Z-amine-A (casein hydrolysate), and proline. Particular examples include media comprising about 0.01 to about 5 milligrams/Liter BAP, about 0.1 to about 5 milligrams/Liter 2,4-D, optionally about 1 to about 50 μM copper, about 0.025 to about 2.5 grams/Liter myo-inositol, about 0.1 to about 10 grams/Liter N—Z-amine-A (casein hydrolysate), about 0.02 to about 2 milligrams/L thiamine-HCl, about 3 to about 300 grams/L maltose and about 0.07 to about 7 grams/Liter proline. For example, a medium can include about 0.5 to about 1 milligrams/L BAP, about 1 milligram/L 2,4-D, about 30 grams/L maltose, about 0.2 milligrams/L thiamine-HCl, optionally about 4.9 μM copper, about 0.25 grams/L myo-inositol, about 1 gram/L N—Z-amine-A (casein hydrolysate), and about 0.7 grams/L proline.

An induction or co-cultivation medium provided herein may further comprise any other appropriate constituents including, without limitation, antioxidants, vitamins (e.g., B₅ vitamins), salts, sorbitol, mannitol, maltose, sucrose, glucose, magnesium chloride, casein hydrosylate, activated charcoal, acetosyringone, and gelling agents such as agar.

In a second culturing step, callus and/or green regenerative tissue can be cultured on a regeneration medium to regenerate a plant. A regeneration medium can promote differentiation of totipotent plant tissues into shoots, roots, and other organized structures and eventually into plantlets that can be transferred to soil. Any appropriate regeneration medium may be used for the practice of the methods of the present invention. In certain embodiments of the invention, the regeneration medium comprises MS (Murashige & Skoog) salts, vitamins (e.g., B₅ vitamins) (about 4.43 g/L), sucrose (about 20 g/L), and myo-inositol (about 1.0 g/L); or MS salts, vitamins (about 4.43 g/L), sucrose (about 30 g/L), and BAP.

As used herein, “plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores. The term “plant tissue” includes differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in a plant or in a plant organ, tissue, or cell culture.

Callus and green regenerative tissues also find use as targets for transformation, e.g., by microprojectile bombardment or infection by Agrobacterium. In some cases, methods for regenerating a plant can further include the step of introducing a nucleic acid into a cell of callus or green regenerative tissue to produce a transformed plant cell. Any appropriate method may be employed to introduce a nucleotide sequence of interest into callus and/or green regenerative tissues. Conventional methods for plant cell transformation include, but are not limited to: (1) microparticle bombardment, (2) Agrobacterium-mediated transformation, (3) microinjection, (4) polyethylene glycol (PEG) procedures, (5) liposome-mediated DNA uptake, (6) electroporation, and (7) vortexing with silica fibers. Particle bombardment has been employed for transformation of a number of plant species, including barley (see, e.g., Wan and Lemaux (1994) Plant Physiol. 104:37-48; Lemaux et al. (1996) BioRad US/EG Technical Bulletin, 2007, 1-6), and corn (see, e.g., Gordon-Kamm et al. (1990) Plant Cell 2(7):603-618). Successful transformation by particle bombardment requires that the target cells are actively dividing, accessible to microprojectiles, culturable in vitro, and totipotent, i.e., capable of regeneration to produce mature fertile or infertile plants.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

In some embodiments, the methods provided herein involve the use of Agrobacterium-mediated gene transfer to produce regenerable plant cells having a nucleotide sequence of interest. Agrobacterium-mediated gene transfer exploits the natural ability of Agrobacterium tumefaciens to transfer DNA (T-DNA) into plant chromosomes. Agrobacterium is a plant pathogen that transfers a set of genes encoded in a region called T-DNA of the Ti plasmid into plant cells at wound sites. The typical result of gene transfer is a tumorous growth called a crown gall in which the T-DNA is stably integrated into a host chromosome. The ability to cause crown gall disease can be removed by deletion of the genes in the T-DNA without loss of DNA transfer and integration. The DNA to be transferred is attached to border sequences that define the end points of an integrated T-DNA.

As used herein, the term “regenerable plant cells comprising a nucleotide sequence of interest” refers to plant cells in which a genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

In some embodiments, methods for producing regenerable plant cells comprising a nucleotide sequence of interest can include the steps of:

(a) culturing a whole plant bud, in vitro-cultured buds, or plantlet in the presence of an induction medium to induce callus or green regenerative tissue formation;

(b) contacting the callus or green regenerative tissue with an Agrobacterium comprising a T-DNA which comprises at least an expression cassette comprising a gene of interest and/or a gene which confers resistance to a selection agent;

(c) co-cultivating the tissue with the Agrobacterium;

(d) selecting regenerable cells comprising the polynucleotide; and

(e) culturing the regenerable cells in the presence of a regeneration medium to produce the transformed plant.

In some embodiments, methods for producing regenerable plant cells comprising a nucleotide sequence of interest can include the steps of:

(a) culturing a whole plant bud, in vitro-cultured buds, or plantlet in the presence of an induction medium to induce callus or green regenerative tissue formation;

(b) contacting the callus or green regenerative tissue with an Agrobacterium comprising a vector which comprises a polynucleotide, wherein the polynucleotide comprises at least an expression cassette comprising a gene of interest and/or a gene which confers resistance to a selection agent;

(c) co-cultivating the tissue with the Agrobacterium;

(d) selecting regenerable cells comprising the polynucleotide; and

(e) culturing the regenerable cells in the presence of a regeneration medium to produce the transformed plant.

In the contacting step, callus and/or green regenerative tissue to be transformed can be contacted to an Agrobacterium. A variety of Agrobacterium species are known in the art, particularly for monocotyledon transformation. Such Agrobacterium can be used in the methods of the invention. See, for example, Hooykaas, P. J. (1989) Plant Mol. Biol. 13:327; Smith et al. (1995) Crop Science 35:301; Chilton, M. O. (1993) Proc. Natl. Acad. Sci. USA 90:3119; Mollony et al. N:Monograph Theor Appl Genet NY, Springer verlag 19:148, 1993; and Ishida et al. (1996) Nature Biotechnol. 14:745; Komari, T. et al. (1996) The Plant Journal 10:165; herein incorporated by reference. See, also, DNA Cloning Service on the world wide web at DNA-cloning.com. In some embodiments an auxotrophic Agrobacterium strain may be used. A variety of auxotrophic Agrobacterium strains are know in the art including but not limited to those described in U.S. Pat. No. 6,323,396, US2010/0186122, and WO2011/068468.

An Agrobacterium strain that is utilized in the methods of the invention can be modified to contain a gene or genes of interest, or a nucleic acid to be expressed in the transformed cells. The nucleic acid to be transferred is incorporated into the T-region and is flanked by T-DNA border sequences. In the Ti plasmid, the T-region is distinct from the vir region whose functions are responsible for transfer and integration. Binary vector systems have been developed where the manipulated disarmed T-DNA carrying foreign DNA and the vir functions are present on separate plasmids. In this manner, a modified T-DNA region comprising foreign DNA (the nucleic acid to be transferred) is constructed in a small plasmid which replicates in E. coli. This plasmid is transferred conjugatively in a tri-parental mating into A. tumefaciens which contains a compatible plasmid-carrying virulence gene. The vir functions are supplied in trans to transfer the T-DNA into the plant genome. Such binary vectors are useful in the practice of the present invention.

The concentration of Agrobacterium that is useful in the methods of the invention may vary depending on the Agrobacterium strain utilized, the tissue being transformed, the plant genotype being transformed, and the like. While the concentration of Agrobacterium may vary, generally a concentration range of about 1×10³ cfu/mL to about 1×10¹⁰ cfu/mL, preferably within the range of about 1×10³ cfu/mL to about 1.5×10⁹ cfu/mL, and still more preferably at about 0.5×10⁹ cfu/mL to about 1.0×10⁹ cfu/mL, will be utilized.

In some cases, the tissue to be contacted with Agrobacterium is callus and/or green regenerative tissue. Callus and/or green regenerative tissue is co-cultivated with Agrobacterium in the presence of a culture medium provided herein. As used interchangeably herein, “co-cultivating”, “co-cultivation”, and “co-culture” refer to incubating Agrobacterium-contacted/infected plant tissue in the presence of the cultivation medium described herein to allow continued T-DNA delivery from Agrobacterium into plant cells.

Callus and/or green regenerative tissue can be co-cultivated with the Agrobacterium for about 1-30 days, preferably about 2-20 days and more preferably about 3-10 days.

In some embodiments, the methods provided herein can further include the step of culturing the tissue (e.g., callus and/or green regenerative tissue) for a length of time prior to the co-cultivation step in a pre-culturing step. “Pre-culturing” and “pre-cultured” as used herein means culturing the cells or tissues in an appropriate pre-culture medium to support plant tissue growth prior to the introduction of a nucleic acid. In some embodiments, tissue is pre-cultured for several days (e.g., about 2 days, about 3 days, about 4 days, about 5 days, or more). Pre-culturing the plant cells may be performed using any method known to one ordinarily skilled in the art. In some cases, pre-culturing can be performed in a cultivation medium as provided herein.

Following the co-cultivation step, the transformed cells may be subjected to an optional resting step. As used herein, “resting” refers to a culture step where plant cells, such as callus and/or green regenerative tissue, or other tissue, are incubated after the introduction of the nucleic acid by Agrobacterium-mediated infection. The resting step permits the preferential initiation and growth of callus from the transformed cells containing the nucleic acid of interest and is usually carried out in the absence of any selective pressures. The transformed plant tissue is subjected to a resting media that typically includes an antibiotic capable of inhibiting Agrobacterium growth. Such antibiotics are known in the art and include cefotaxime, timetin, vancomycin, carbenicillin, Plant Preservative Mixture™ (Plant Cell Technology, Inc., Washington, D.C.), and the like. Other antibiotics include, without limitation, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, and vancomycin. Concentrations of the antibiotic will vary according to what is standard for each antibiotic. For example, concentrations of carbenicillin will range from about 50 mg/L to about 250 mg/L, carbenicillin in solid media, preferably about 75 mg/L to about 200 mg/L, and more preferably about 100-125 mg/L. Those of ordinary skill in the art of monocot transformation will recognize that the concentration of antibiotic can be optimized for a particular transformation protocol without undue experimentation.

In some embodiments, the resting phase cultures are allowed to rest in the dark or under dim light conditions (5-30 μmole/m⁻²/s⁻¹) at 28° C. for about 1 to about 15 days, preferably for about 3 to about 10 days, more preferably for about 3 to about 7 days.

Where no resting step is used, an extended co-cultivation step can be used to provide a period of culture time prior to the addition of a selective agent for the transformed cells.

The methods provided herein further include selecting regenerable cells comprising a nucleotide sequence of interest. “Selecting” as used herein refers to the culture step in plant transformation where the transformed cells that have received and are expressing a selection marker from the introduced nucleic acid are selected. Following the co-cultivation step, or following the resting step, where it is used, the transformed cells can be exposed to selective pressure to select for those cells that have received and are expressing polypeptide from the heterologous nucleic acid introduced by Agrobacterium. In some cases, cells may be exposed to a selective pressure in order to favor those cells that express the selection marker and may include the use of a selective agent that allows for selection of transformants containing at least one selection marker insert. For example, where the cells are embryos, the embryos can be transferred to plates with solid medium that includes both an antibiotic to inhibit growth of the Agrobacterium and a selection agent. The selection agent used to select for transformants will select for preferential growth of explants containing at least one selectable marker insert positioned within the superbinary vector and delivered by the Agrobacterium.

As indicated above, any suitable selection marker may be used including, without limitation, bar, pat, als, GAT, PMI, hpt, nptII and positive and/or negative selectable markers and visible selection marker genes such as DS-RED, GFP, CFP, YFP, GUS and the like. Any suitable selective agent or compound may be used including, without limitation, herbicides, such as, bialaphos, glufosinate-NH4 (PPT), glyphosate, sugar, such as mannose, and antibiotics such as hygromycin B, kanamycin, paromomycin, or G418, and the like. Visual markers themselves can also be used to identify transgenic events without using any selective agents. During the selecting step, dead and necrotic tissues can be discarded, and surviving callus, green shoots, and shoot buds can be subcultured to fresh medium containing the selection agent.

Selecting may optionally be carried out in light, dim, or dark conditions. The length of exposure of the plant cell to light, dim, or dark conditions may vary based in part on the type of plant species and genotype being transformed. Preferably, plant cells are rested and selected in dark conditions.

After transformed plant tissue has been identified and selected, the transformed tissue can be regenerated into whole plants. The regenerated plants can be fertile or infertile. Any appropriate method of regenerating whole plants can be used. The regeneration, development, and cultivation of plants from various transformed explants are well known in the art. See, for example, McCormick et al. (1986) Plant Cell Reports 5:81-84; Weissbach and Weissbach, In: Methods for Plant Molecular Biology, (Eds.), Academic Press, Inc. San Diego, Calif., (1988). This regeneration and growth process typically includes the steps of selection of transformed cells, culturing those individualized cells through the usual stages of embryonic development through the rooted plantlet stage.

In some cases, transformed callus and/or green regenerative tissue or other transformed plant tissue can be subcultured at regular or irregular intervals in the same medium. Individual calli can be individually separated to ensure that only one whole plant is regenerated per callus and, therefore, that all regenerated plants are derived from independent transformation events. The resulting transgenic rooted shoots are thereafter planted in an appropriate plant growth medium such as soil.

For vegetatively propagated plants such as sugarcane, conventional propagation or micropropagation (i.e., tissue culture) methods can be performed to produce additional generations or “clones” from regenerated plants.

In some cases, the regenerated plants are self-pollinated to provide homozygous transgenic plants. Otherwise, pollen obtained from the regenerated plants can be crossed to seed-grown plants of agronomically important lines. Conversely, pollen from plants of these important lines is used to pollinate regenerated plants. Two or more generations may be grown to ensure that expression of the desired phenotypic characteristic is stably maintained and inherited and then seeds harvested to ensure expression of the desired phenotypic characteristic has been achieved. In this manner, the present invention can provide transformed seed (also referred to as “transgenic seed”) having a polynucleotide of the invention, for example, an expression cassette of the invention, stably incorporated into their genome.

The methods described herein provide for an efficient method of increasing the transformation of monocots. Any suitable monocot may be used with the methods and compositions described herein. These include, without limitation, sugarcane (e.g., Saccharum spp.), corn (Zea mays), wheat (Triticum aestivum), rice (Oryza sativa), rye (Secale cereale), sorghum (Sorghum bicolor, Sorghum vulgare), and the like.

The methods of the invention involve introducing a polynucleotide into a plant. “Introducing” is intended to mean presenting to the plant the polynucleotide in such a manner that the polynucleotide gains access to the interior of a cell of the plant. The methods of the invention do not depend on a particular method for introducing a polynucleotide into a plant, only that the polynucleotide gains access to the interior of at least one cell of the plant. Methods for introducing polynucleotide into plants are known in the art including, but not limited to, stable transformation methods, transient transformation methods, and virus-mediated methods.

“Stable transformation” is intended to mean that the nucleotide construct or polynucleotide introduced into a plant integrates into the genome of the plant and is capable of being inherited by the progeny thereof “Transient transformation” is intended to mean that a polynucleotide is introduced into the plant and does not integrate into the genome of the plant or a polypeptide is introduced into a plant.

The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotides of interest can be provided in expression cassettes for expression in the plant of interest, particularly sugarcane. The cassette will include 5′ and 3′ regulatory sequences operably linked to a polynucleotide of the interest. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a polynucleotide of the interest, and a transcriptional and translational termination region (i.e., termination region) functional in plants. The regulatory regions (i.e., promoters, transcriptional regulatory regions, and translational termination regions) and/or the polynucleotide of interest may be native/analogous to the host cell or to each other. Alternatively, the regulatory regions and/or the polynucleotide of the interest may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the sequences using heterologous promoters, the native promoter sequences may be used. Such constructs can change expression levels of the polynucleotide of interest and/or the protein encoded thereby in the plant or plant cell. Thus, the phenotype of the plant or plant cell can be altered.

The termination region may be native with the transcriptional initiation region, may be native with the operably linked polynucleotide of interest, may be native with the plant host, or may be derived from another source (i.e., foreign or heterologous) to the promoter, the polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention, including the native promoter of the polynucleotide sequence of interest. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1 :961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of a polynucleotide of interest within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed; herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (nptII) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

The methods disclosed herein are useful in regulating expression of any heterologous nucleotide sequence in a host plant in order to vary the phenotype of a plant. As will be evident to one of skill in the art, any nucleic acid of interest can be used in the methods of the invention. For example, a sugarcane plant can be engineered to express disease and insect resistance genes, genes conferring nutritional value, genes to confer male and/or female sterility, antifungal, antibacterial or antiviral genes, and the like. Likewise, the method can be used to transfer any nucleic acid to control gene expression. For example, the nucleic acid to be transferred could encode an antisense oligonucleotide.

Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, and commercial products.

Agronomically important traits such as oil, protein content, and the like can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene); glyphosate (e.g., the EPSPS gene and the GAT gene; see, for example, U.S. Publication No. 20040082770 and WO 03/092360); or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

In some embodiments, a nucleic acid to be introduced according to the methods provided herein can comprise a developmental gene cassette. For example, the polynucleotide can comprise a developmental gene cassette encoding a cell proliferation transcription factor such as a babyboom (BBM) or Wuschel (WUS). See, for example, U.S. Patent Application Publication No. 2011/0167516, which is herein incorporated by reference in its entirety. The BBM gene encodes a cell proliferation factor which is a member of the AP2 family of transcription factors. The Arabidopsis BBM (AtBBM, the polynucleotide and amino acid sequence of which is set forth in SEQ ID NO: 1 and 2) is preferentially expressed in the developing embryo and seeds and has been shown to play a central role in regulating embryo-specific pathways. Overexpression of AtBBM has been shown to induce spontaneous formation of somatic embryos and cotyledon-like structures on seedlings. See Boutiler et al. (2002) The Plant Cell 14:1737-1749. Maize BBM (the polynucleotide and amino acid sequence of which is set forth in SEQ ID NO: 3 and 4) also induces embryogenesis and promotes transformation (see U.S. Pat. No. 7,579,529, which is herein incorporated by reference in its entirety). Thus, BBM polypeptides stimulate proliferation, induce embryogenesis, enhance the regenerative capacity of a plant, enhance transformation, and as demonstrated herein, enhance rates of targeted polynucleotide modification. As used herein “regeneration” refers to a morphogenic response which results in the production of new tissues, organs, embryos, whole plants or parts of whole plants that are derived from a single cell or a group of cells. Regeneration may proceed indirectly via a callus phase or directly, without an intervening callus phase. “Regenerative capacity” refers to the ability of a plant cell to undergo regeneration.

In other embodiments, other cell proliferation factors, such as, Lec1, Kn1 family, WUSCHEL (e.g., WUS1, the polynucleotide and amino acid sequence of which is set forth in SEQ ID NO: 5 and 6; WUS2, the polynucleotide and amino acid sequence of which is set forth in SEQ ID NO: 7 and 8; WUS2 alt, the polynucleotide and amino acid sequence of which is set forth in SEQ ID NO: 9 and 10; WUS3, the polynucleotide and amino acid sequence of which is set forth in SEQ ID NO: 11 and 12), Zwille, and Aintegumeta (ANT), may be used alone, or in combination with a BBM polypeptide or other cell proliferation factor. See, for example, U.S. Application Publication No. 2003/0135889, International Application Publication No. WO 03/001902, and U.S. Pat. No. 6,512,165, each of which is herein incorporated by reference. When multiple cell proliferation factors are used, or when a BBM polypeptide is used along with any of the aforementioned polypeptides, the polynucleotides encoding each of the factors can be present on the same expression cassette or on separate expression cassettes. When two or more factors are coded for by separate expression cassettes, the expression cassettes can be provided to the plant simultaneously or sequentially.

Many, if not all, of the steps of methods described herein can be mechanized and/or at least substantially automated. Mechanized or automated steps could include, without limitation, handling plants and plant tissues, micropropagating plantlets, preparing and/or maintaining in vitro cultured buds, handling calli or green regenerative tissue, and/or performing transformation methods. Mechanized or automated steps could further include post-processing steps including testing, evaluation, and storage or recording of the results of the same.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

The following examples are presented by way of illustration, and not by way of limitation.

### EXAMPLES

### Example 1

### Tissue Culture System for Callus and Green Regenerative Tissue Induction

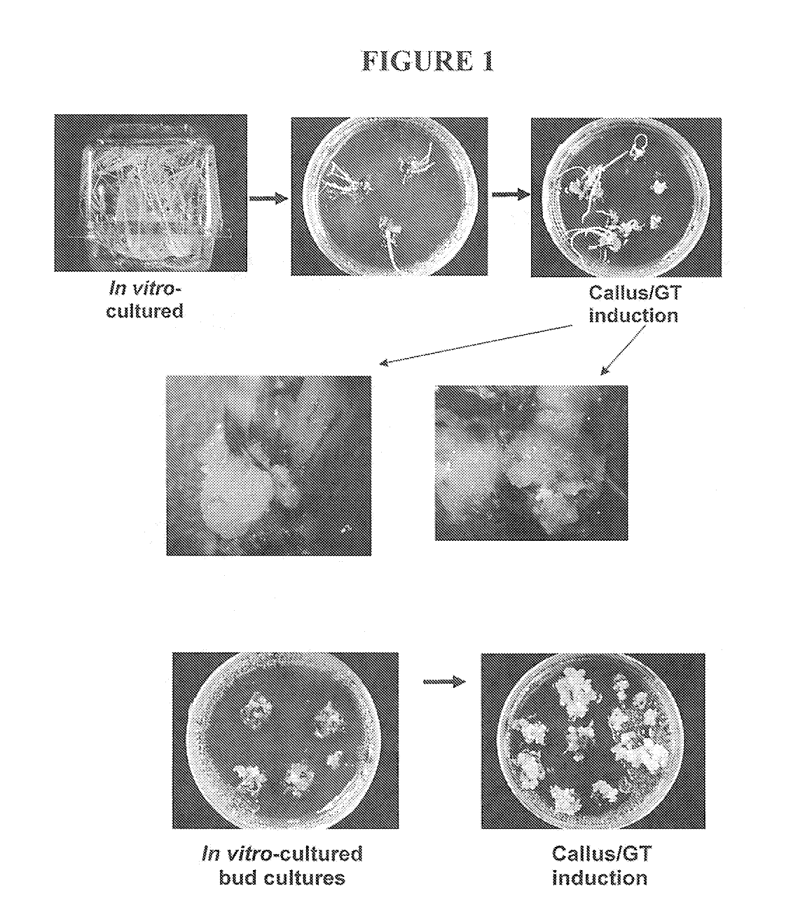

Callus and green regenerative tissues were the target explants for transformation. The target tissues can be derived from in vitro-cultured whole plantlets, in vitro cultured buds, and immature whorls. Plantlets (about 6-8 plantlets) were in sugarcane maintenance media MSA in Phytatrays (FIG. 1) or Magenta boxes. MSA+100 mg/L carbenicillin can be used to control contamination of endogenous bacteria if the source material is not completely sterile. In vitro-cultured buds were obtained from cultivars CP96-1252, CP01-1372, CP89-2376, and CPCL97-2730. Approximately 1-3 plantlets or in vitro cultured buds with approximately 3-5 tillers/buds were separated as necessary, making sure to preserve the leaf base and meristem regions. Plantlets and bud pieces (about 3-6 pieces per plate) were transferred in a tissue induction medium. Nine different tissue induction media were tested for the ability of each sugarcane cultivar to induce callus or green regenerative tissue: EM3D, EM3DC, EM3DBC1, EM3DBC2, EM3DBC3, DBC1, DBC2, DBC3, and DBC4 media. Removal of some leaf blades and roots from whole plantlets may increase induction efficiency. Tissues were incubated at 26-28° C. under dark or dim light (0-10 μmol M⁻² sec⁻¹) conditions.

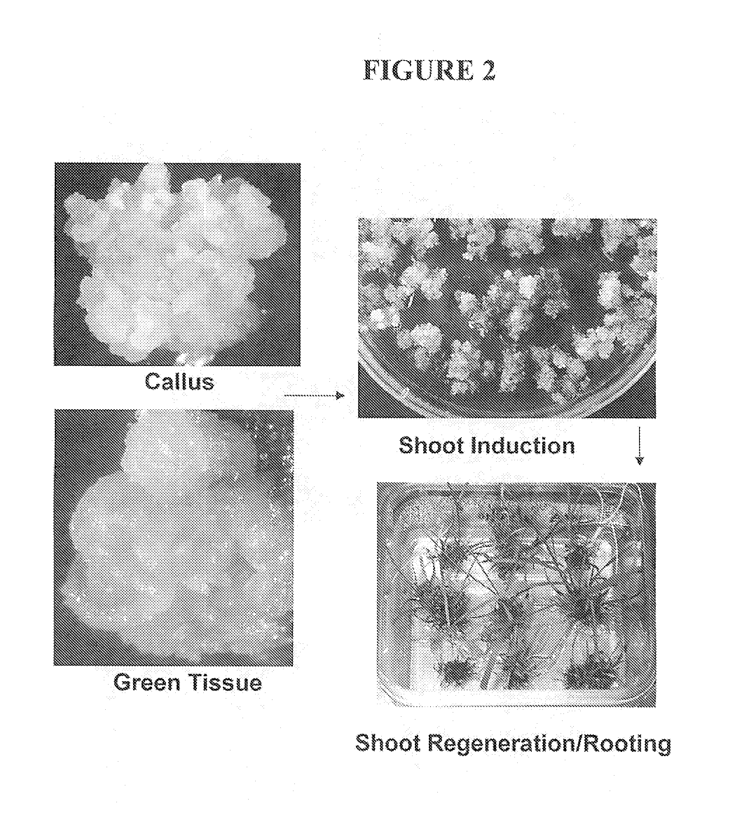

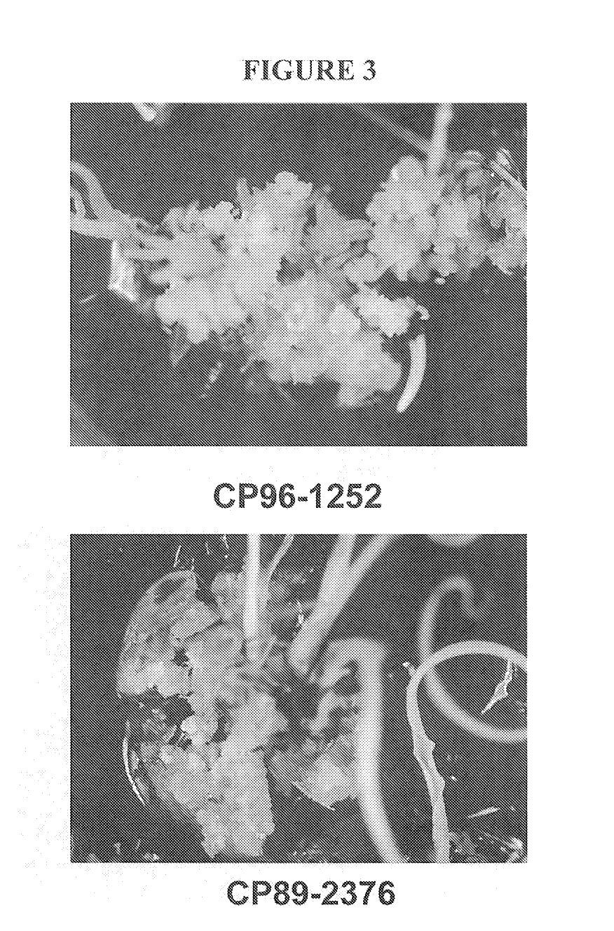

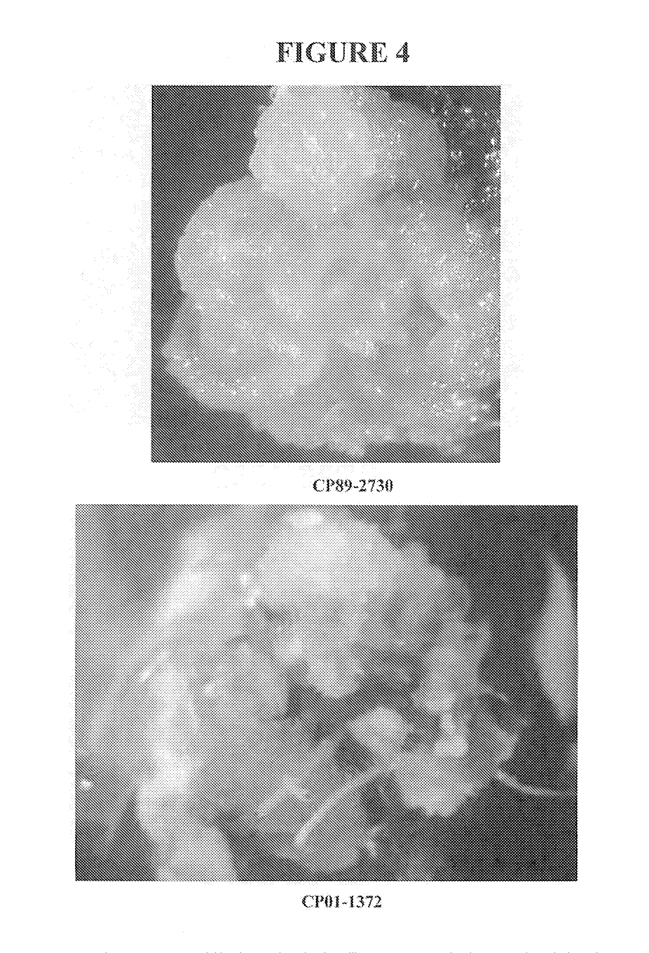

After 2-4 weeks, callus and green regenerative tissues were induced mostly from the base (crown) of the plantlet and some parts of in vitro cultured buds (FIGS. 1, 2, and 3). Both whole plantlets and in vitro cultured buds were efficient in inducing quality tissues. Only white, compact embryogenic callus (FIGS. 1-3) or light green tissue (FIGS. 2 and 4) were isolated and transferred to fresh media; a much higher proportion of callus tissue was induced than green tissue. Tissue with a “watery” morphology was not transferred because this type of tissue was not efficient in regeneration. Tissues were broken into small pieces, about 2-3 mm in diameter. Plates were sealed and incubated at 26-28° C. under dark or dim light conditions for 3-4 weeks until there was enough mass of tissue. Proliferating and maintaining white, compact embryogenic callus or light green tissue were continued every 3 weeks onto fresh media as necessary. Tissues were tested for regeneration in MSB medium (FIG. 2).

Each cultivar had slightly different response to different tissue induction media, but all 4 cultivars (CP96-1252, CP01-1372, CP89-2376, and CPCL97-2730) tested showed good tissue induction on all 9 induction media (Table 1). The lower BAP level in the medium, the higher percentage of white embyrogenic cultures; the higher BAP level in the medium, the higher percentage of green organogenic cultures. DBC3 medium was, in general, optimal for all 4 cultivars tested.

TABLE 1
Tissue Culture Response of Four Sugarcane Cultivars

| | Sugarcane Cultivar | | | |
|---|---|---|---|---|
| | CP96-1252 | CP01-1372 | CP89-2376 | CPCL97-2730 |
| Tissue | +++ | +++++ | +++(+) | ++++ |
| Culture | | | | |
| Response | | | | |

### Media for Tissue Culture:

EM3D contains MS salts and vitamins (4.43 g/L) plus sucrose (20 g/L); N—Z-amine-A (casein hydrolysate) (0.5 g/L); coconut H₂O (100 mL/L); 2,4-D (3.0 mg/L); Adjust volume to 1 L with ddH₂O; pH 5.8—Adjust pH with 1 M KOH; Phytagel (3.5 g/L); autoclave.

EM3DC contains MS salts and vitamins (4.43 g/L) plus sucrose (20 g/L); N—Z-amine-A (casein hydrolysate) (0.5 g/L); coconut H₂O (100 mL/L); CuSO₄ (4.9 μM); 2,4-D (3.0 mg/L); Adjust volume to 1 L with ddH₂O; pH 5.8—Adjust pH with 1 M KOH; Phytagel (3.5 g/L); autoclave.

EM3DBC1 contains MS salts and vitamins (4.43 g/L) plus sucrose (20 g/L); N—Z-amine-A (casein hydrolysate) (0.5 g/L); coconut H₂O (100 mL/L); CuSO₄ (4.9 μM); 2,4-D (3.0 mg/L); BAP (0.01 mg/L); Adjust volume to 1 L with ddH₂O; pH 5.8—Adjust pH with 1 M KOH; Phytagel (3.5 g/L); autoclave.

EM3DBC2 contains MS salts and vitamins (4.43 g/L) plus sucrose (20 g/L); N—Z-amine-A (casein hydrolysate) (0.5 g/L); coconut H₂O (100 mL/L); CuSO₄ (4.9 μM); 2,4-D (3.0 mg/L); BAP (0.1 mg/L); Adjust volume to 1 L with ddH₂O; pH 5.8—Adjust pH with 1 M KOH; Phytagel (3.5 g/L); autoclave.

EM3DBC3 contains MS salts and vitamins (4.43 g/L) plus sucrose (20 g/L); N—Z-amine-A (casein hydrolysate) (0.5 g/L); coconut H₂O (100 mL/L); CuSO₄ (4.9 μM); 2,4-D (1.0 mg/L); BAP (0.5 mg/L); Adjust volume to 1 L with ddH₂O; pH 5.8—Adjust pH with 1 M KOH; Phytagel (3.5 g/L); autoclave.

DBC1 contains MS salts (4.3 g/L) plus maltose (30 g/L); thiamine-HCI (1 mg/mL); myo-inositol (0.25 g/L); N—Z-amine-A (casein hydrolysate) (1 g/L); proline (0.69 g/L); CuSO₄ (4.9 μM); 2,4-D (2.5 mg/L); BAP (0.01 mg/L); Adjust volume to 1 L with ddH₂O; pH 5.8—Adjust pH with 1 M KOH; Phytagel (3.5 g/L); autoclave.

DBC2 contains MS salts (4.3 g/L) plus maltose (30 g/L); thiamine-HC1 (1 mg/mL); myo-inositol (0.25 g/L); N—Z-amine-A (casein hydrolysate) (1 g/L); proline (0.69 g/L); CuSO4 (4.9 μM); 2,4-D (2.5 mg/L); BAP (0.1 mg/L); Adjust volume to 1 L with ddH₂O; pH 5.8—Adjust pH with 1 M KOH; Phytagel (3.5 g/L); autoclave.

DBC3 contains MS salts (4.3 g/L) plus maltose (30 g/L); thiamine-HC1 (1 mg/mL); myo-inositol(0.25 g/L); N—Z-amine-A (casein hydrolysate) (1 g/L); proline (0.69 g/L); CuSO₄ (4.9 μM); 2,4-D (1.0 mg/L); BAP (0.5 mg/L); Adjust volume to 1 L with ddH₂O; pH 5.8—Adjust pH with 1 M KOH; Phytagel (3.5 g/L); autoclave.

DBC4 contains MS salts (4.3 g/L) plus maltose (30 g/L); thiamine-HC1 (1 mg/mL); myo-inositol(0.25 g/L); N—Z-amine-A (casein hydrolysate) (1 g/L); proline (0.69 g/L); CuSO₄ (4.9 μM); 2,4-D (1.0 mg/L); BAP (1.0 mg/L); Adjust volume to 1 L with ddH₂O; pH 5.8—Adjust pH with 1 M KOH; Phytagel (3.5 g/L); autoclave.

MSA contains MS salts and vitamins (4.43 g/L) plus sucrose (20 g/L); myo-inositol (1.0 g/L); Adjust volume to 1 L with ddH₂O; pH 5.8—Adjust pH with 1 M KOH; Phytagel (3.5 g/L); autoclave.

### Example 2

### Sugarcane Agrobacterium Transformation Using Sugarcane Callus and Green Regenerative Tissues

### Media for Plant Transformation:

Liquid DBC3(M5G) contains MS salts (4.3 g/L) plus maltose (30 g/L); glucose (5 g/L); thiamine-HC1 (1 mg/mL); myo-inositol (0.25 g/L); N—Z-amine-A (casein hydrolysate) (1 g/L); proline (0.69 g/L); CuSO4 (4.9 μM); 2,4-D (1.0 mg/L); BAP (0.5 mg/L); Adjust volume to 1 L with ddH2O; pH 5.8—Adjust pH with 1 M KOH; autoclave.

DBC3 contains MS salts (4.3 g/L) plus maltose (30 g/L); thiamine-HC1 (1 mg/mL); myo-inositol (0.25 g/L); N—Z-amine-A (casein hydrolysate) (1 g/L); proline (0.69 g/L); CuSO₄ (4.9 μM); 2,4-D (1.0 mg/L); BAP (0.5 mg/L); Adjust volume to 1 L with ddH₂O; pH 5.8—Adjust pH with 1 M KOH; Phytagel (3.5 g/L); autoclave.

DBC6 contains MS salts (4.3 g/L) plus maltose (30 g/L); thiamine-HC1 (1 mg/mL); myo-inositol (0.25 g/L); N—Z-amine-A (casein hydrolysate) (1 g/L); proline (0.69 g/L); CuSO₄ (4.9 μM); 2,4-D (0.5 mg/L); BAP (2.0 mg/L); Adjust volume to 1 L with ddH₂O; pH 5.8—Adjust pH with 1 M KOH; Phytagel (3.5 g/L); autoclave.

MSB contains MS salts and vitamins (4.43 g/L) plus sucrose (20 g/L); myo-inositol (1.0 g/L); indole-3-butyric acid (IBA, 0.5 mg/L); Adjust volume to 1 L with ddH₂O; pH 5.8—Adjust pH with 1 M KOH; Phytagel (3.5 g/L); autoclave.

### Preparation of Agrobacterium Suspension:

Agrobacterium tumefaciens harboring a binary vector from a −80° frozen aliquot was streaked out onto solid PHI-L or LB medium containing an appropriate antibiotic and culture at 28° C. in the dark for 2-3 days. A single colony or multiple colonies were picked from the master plate and streaked onto a plate containing PHI-M medium and incubated at 28° C. in the dark for 1-2 days. Agrobacterium cells were collected from the solid medium using 5 mL 10 mM MgSO₄ medium (Agrobacterium infection medium) plus 100 μM acetosyringone. One mL of the suspension was transferred to a spectrophotometer tube and the OD of the suspension to 0.35-0.40 at 550 nm using the same medium.

### Agrobacterium Infection and Co-Cultivation:

Good quality callus/green tissues were collected in an empty Petri dish and exposed to air in the hood for about 30 minutes. Tissue that is younger than 2 months old is considered ideal for transformation. One mL Agrobacterium suspension was added to the Petri dish, the tissues were broken or chopped into small pieces and additional 1-3 mL Agrobacterium suspension was then added to cover all the tissues. The Petri dish was placed into a transparent polycarbonate desiccator container, covered the desiccator container and connected to an in-house vacuum system for 20 minutes. After infection, the Agrobacterium suspension was drawn off from the Petri dish and the tissues were transferred onto 2 layers of VWR 415 filter paper (7.5 cm diameter) of a new Petri dish and 0.7-2.0 mL liquid DBC3 (M5G) medium plus 100 μM acetosyringone was added depending tissue amount for co-cultivation. The top layer of filter paper containing the infected tissues was transferred to a fresh layer of filter paper of another new Petri dish. The infected tissues wad incubated at 21° C. in the dark for 3 days.

### Selection and Plant Regeneration:

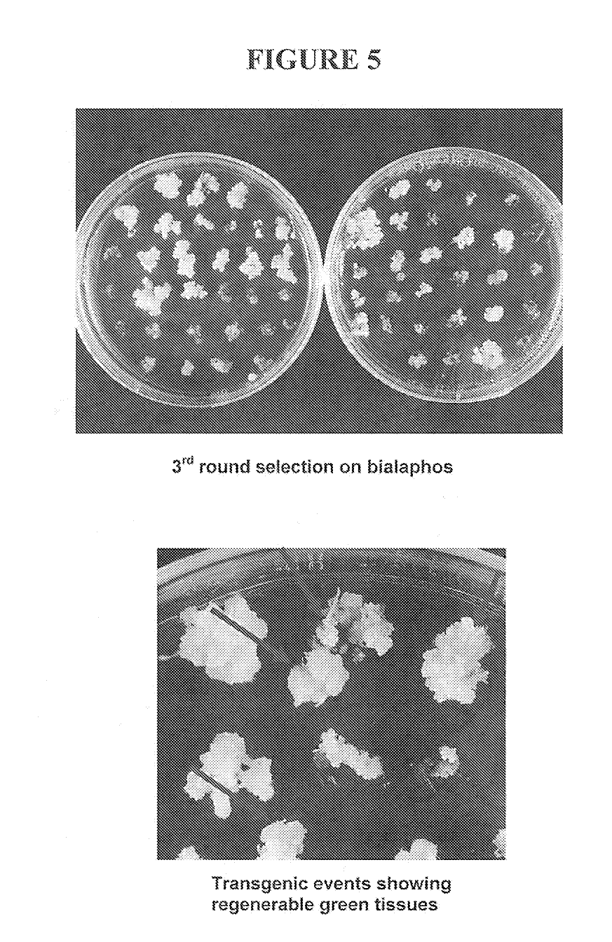

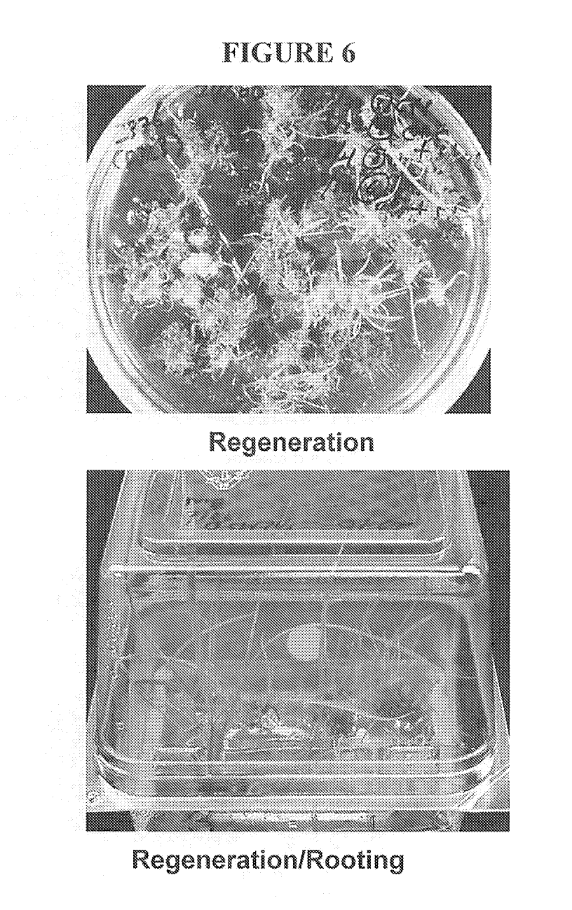

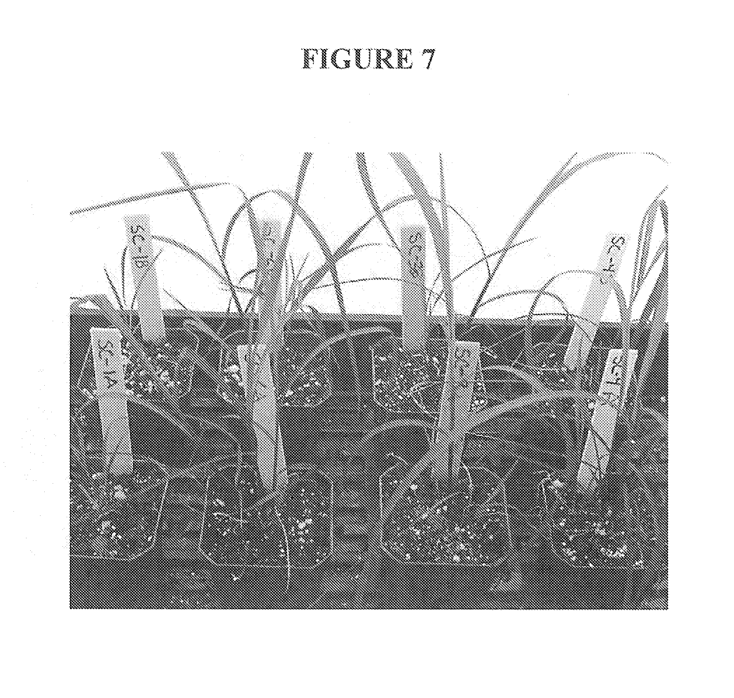

Callus or green regenerative tissues were transferred to first round selection DBC3 containing antibiotics (timentin and cefotaxime) and 3 mg/L bialaphos (Meiji Seika, Tokyo, Japan). Tissues were transferred to 2nd round selection DBC6 containing antibiotics and 3-5 mg/L bialaphos and subcultured for 3 weeks at 26-28° C. in dark or dim light conditions (FIG. 5). At the 3rd round selection on DBC6 medium containing antibiotics and bialaphos, tissues were broken into smaller pieces and exposed to bright light conditions (30-150 μmol M⁻² sec⁻¹) for 2-3 weeks. Shoot elongated tissues were broken into small pieces and transferred to MSB regeneration/rooting medium containing antibiotics and 3 mg/L bialaphos (FIG. 6). Single plantlets were separated and transferred to soil (FIG. 7).

### Confirmation of Transgenic Events:

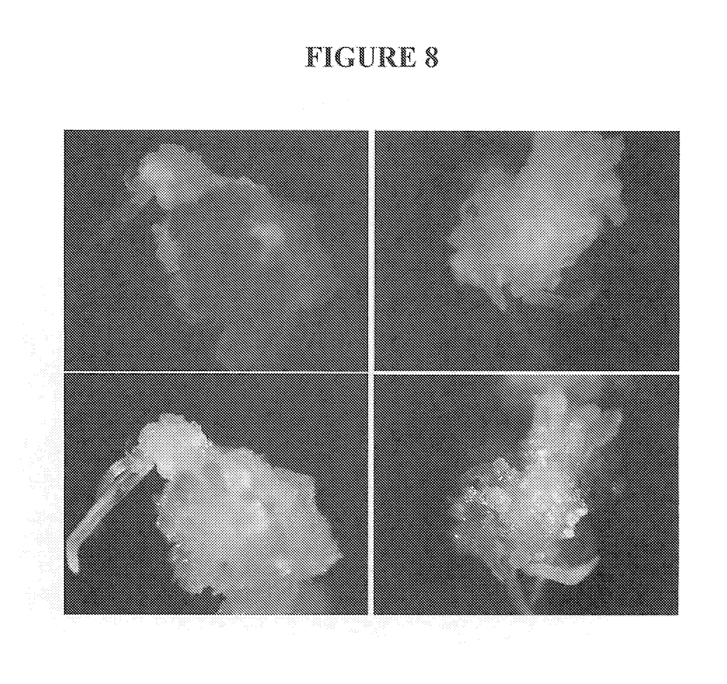

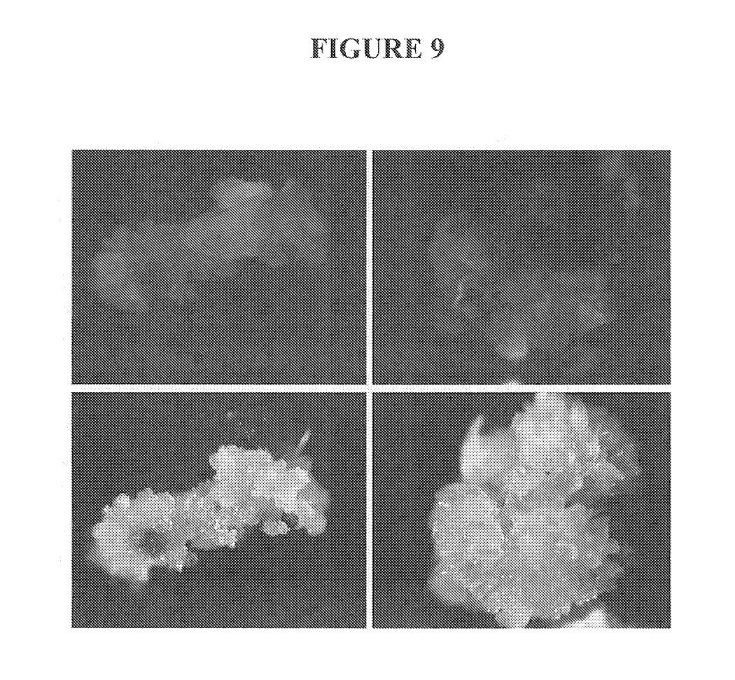

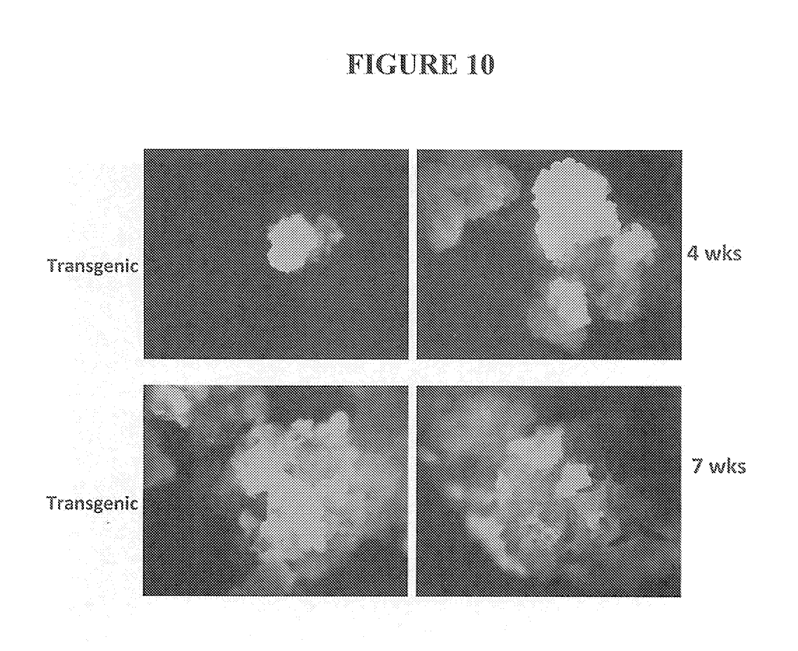

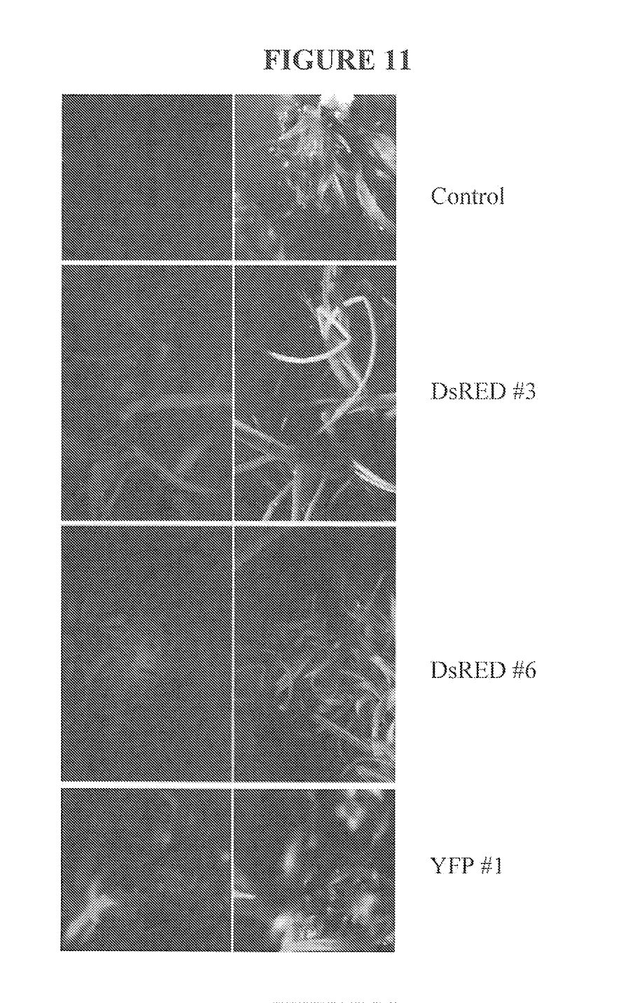

The putative stable callus/green tissues/regenerating plants were identified based on the visible marker gene (RFP, CFP or YFP) expression (FIGS. 8, 9, 10, and 11). All of these putative transgenic callus tissues were transferred to medium for plant regeneration under standard regeneration conditions. The final confirmation of stable transformation frequency is determined based on molecular analysis such as PCR and Southern blot hybridization.

### Example 3

### Agrobacterium Strain Comparison for Sugarcane Transformation

Two Agrobacterium strains, AGL1 and LBA4404, were compared for transformation frequency in CP89-2376 and CP01-1372. Callus tissues of both cultivars were induced and maintained on DBC3 medium. Tissues were infected with Agrobacterium containing pDsREDmoPAT in liquid 10 mM MgSO₄ plus 100 μM acetosyringone and broken/chopped into small pieces. The tissues were then co-cultivated with liquid DBC3 (M5G) medium plus 100 μM acetosyringone on the filter paper at 21° C. in the dark. Three days after co-cultivation, the tissues were transferred to DBC3+100 mg/L cefotaxime+150 mg/L timentin for AGL1 and DBC3+100 mg/L carbenicillin for LBA4404, and incubated at 26° C. (±1° C.) in the dark or dim light for 3-7 days. The tissues were then transferred to the same media as the previous step plus 3 or 5 mg/L bialaphos. After 2 to 3 weeks, the tissues were transferred to 2nd round selection DBC6 containing antibiotics and 3-5 mg/L bialaphos. After two months from the initiation of the experiment, transgenic callus events were cultured for plant regeneration on the same regeneration medium (MSB) plus 1 or 3 mg/L bialaphos at the regular regeneration conditions. Transformation frequency was calculated by the number of explants producing transgenic plants divided by the number of explants infected by Agrobacterium. Table 2 demonstrates that transformation frequency in CP89-2376 was much higher using AGL1 than LBA4404 in these experiments. However, CP01-1372 was very recalcitrant in transformation using either of the two Agrobacterium strains.

TABLE 2
Transformation Frequency in Sugarcane Using
Two Agrobacterium Strains

| | Sugarcane Cultivar | | | |
|---|---|---|---|---|
| | CP89-2376 | | CP01-1372 | |
| | Agrobacterium Strain | | | |
| | AGL1 | LBA4404 | AGL1 | LBA4404 |
| Expt 1 | 62.5% (5/8) | 50.0% (4/8) | 0% (0/8) | 0% (0/8) |
| Expt 2 | 87.5% (7/8) | 0% (0/8) | 0% (0/8) | 0% (0/8) |
| Average | 75.0% (12/16) | 25.0% (4/16) | 0% (0/16) | 0% (0/16) |

### Example 4

### Tissue Type Comparison for Sugarcane Transformation

Callus and green regenerative tissues were compared for transformation frequency in CP89-2376. Both callus tissue induced and maintained on DBC3 medium while green tissue was induced on DBC3 or DBC4 and then maintained on DBC4 or DBC6 medium. Both tissues were infected with Agrobacterium AGL1 containing pVERYFPmoPAT in liquid 10 mM MgSO₄ plus 100 μM acetosyringone and broken/chopped into small pieces. The tissues were then co-cultivated with liquid DBC3 (M5G) medium plus 100 μM acetosyringone on the filter paper at 21° C. in the dark. Three days after co-cultivation the tissues were transferred to DBC3 and DBC6+100 mg/L cefotaxime+150 mg/L timentin, and incubated at 26° C. (±1° C.) in the dark or dim light for 3-7 days. The tissues were then transferred to the same media as the previous step plus 3 or 5 mg/L bialaphos. After 2 to 3 weeks, the tissues were transferred to 2nd round selection DBC6 containing antibiotics and 3-5 mg/L bialaphos. After two months from the initiation of the experiment, transgenic callus events were cultured for plant regeneration on MSB regeneration medium plus 1 or 3 mg/L bialaphos at the regular regeneration conditions. Transformation frequency was calculated by the number of explants producing transgenic plants divided by the number of explants infected by Agrobacterium. Table 3 and FIG. 5 demonstrated that transformation frequency was even higher using callus tissue as a transformation target than green regenerative tissue in these experiments.

TABLE 3
Transformation Frequency in Sugarcane (cv. CP89-2376) Using
Callus and Green Regenerative Tissues as Transformation Targets

| | Callus tissue | Green regenerative tissue |
|---|---|---|
| Expt1 | 87.5% (7/8) | 12.5% (1/8) |
| Expt 2 | 150.0% (12/8) | 25.0% (2/8) |
| Expt 3 | 62.5% (5/8) | 0% (0/8) |
| Average | 100.0% (24/24) | 12.5% (3/24) |

### Example 5

### Sugarcane Germplasm Screening for T-DNA Delivery and Stable Sugarcane Transformation

Callus tissues of seven different United States sugarcane cultivars (CP96-1252, CP01-1372, CP89-2376, CPCL97-2730, HoCP85-845, CP89-2143 and CP88-1762) induced and maintained on DBC3 medium. Eight pieces of tissues 0.4-0.5 cm in size per treatment were infected with Agrobacterium AGL1 containing pDsREDmoPAT or pVERYFPmoPAT in liquid 10 mM MgSO4 plus 100 uM acetosyringone and broken/chopped into small pieces. The tissues were then co-cultivated with liquid DBC3 (M5G) medium plus 100 uM acetosyringone on the filter paper at 21° C. in the dark. Three days after co-cultivation the tissues were transferred to DBC3+100 mg/L cefotaxime+150 mg/L timentin, and incubated at 26° C. (±1° C.) in the dark or dim light for 3-7 days. DsRED or YFP expression was monitored using fluorescence microscope. The tissues were then transferred to the same media as the previous step plus 3 or 5 mg/L bialaphos. After 2 to 3 weeks, the tissues were transferred to 2nd round selection DBC6 containing antibiotics and 3-5 mg/L bialaphos. After two months from the initiation of the experiment, transgenic callus events were cultured for plant regeneration on MSB regeneration medium plus 1 or 3 mg/L bialaphos at the regular regeneration conditions. Transformation frequency was calculated by the number of explants producing transgenic plants divided by the number of explants infected by Agrobacterium.

T-DNA delivery efficiency, observed at 3 to 7 days after Agrobacterium infection, was dependent on cultivars. Out of 7 cultivars tested, CP89-2376 and CP88-1762 showed more DsRED- or YFP-expressing foci than CP96-1252, CP01-1372, CPCL97-2730, HoCP85-845 and CP89-2143 (Table 4).

TABLE 4
T-DNA Delivery Efficiency in Callus Tissues of Seven U.S. Sugarcane Cultivars

| | Sugarcane Cultivar | | | | | | |
|---|---|---|---|---|---|---|---|
| | CP96-1252 | CP01-1372 | CP89-2376 | CPCL97-2730 | HoCP85-845 | CP89-2143 | CP88-1762 |
| T-DNA | +(+) | + | +++ | + | + | +(+) | +++ |
| Delivery | | | | | | | |

CP89-2376 and CP88-1762 showed highest stable transformation frequencies out of 7 sugarcane cultivars tested. Both cultivars had >100% transformation frequencies while no transgenic events were obtained from the remaining 5 cultivars, CP96-1252, CP01-1372, CPCL97-2730, HoCP85-845 and CP89-2143 (Table 5). T-DNA delivery efficiency was highly correlated with stable transformation frequency in all 7 cultivars tested (Tables 4 and 5).

TABLE 5
Transformation Frequency in Seven Sugarcane Cultivars

| CP96-1252 | CP01-1372 | CP89-2376 | CPCL97-2730 | HoCP85-845 | CP89-2143 | CP88-1762 |
|---|---|---|---|---|---|---|
| n.t.* | n.t. | 75.0% | n.t. | n.t. | n.t. | n.t. |
| | | (6/8) | | | | |
| 0% | 0% | 100.0% | 0% | n.t. | n.t. | n.t. |
| (0/8) | (0/8) | (8/8) | (0/8) | | | |
| n.t. | n.t. | 87.5% | n.t. | n.t. | n.t. | n.t. |
| | | (7/8) | | | | |
| n.t. | n.t. | 150.0% | n.t. | 0% | n.t. | n.t. |
| | | (12/8) | | (0/8) | | |
| n.t. | n.t. | n.t. | n.t. | n.t. | 0% | 62.5% |
| | | | | | (0/8) | (5/8) |
| n.t. | n.t. | 100.0% | n.t. | n.t. | 0% | 137.5% |
| | | (8/8) | | | (0/8) | (11/8) |
| n.t. | n.t. | 187.5% | n.t. | n.t. | n.t. | 137.5% |
| | | (15/8) | | | | (11/8) |

n.t.* not tested

### Example 6

### Sugarcane Tissue Culture and Transformation Protocol

A. Callus and Green Regenerative Tissue Induction and Proliferation from In Vitro-Cultured Whole Plantlets and In Vitro-Cultured Buds

Plantlets (about 6-8 plantlets) were maintained in sugarcane maintenance media MSA in Phytatrays (Sigma Aldrich Brand Phytatray II). MSA+100 mg/L carbenicillin (ICN, Costa Mesa, Calif.) may also be used to control contamination of endogenous bacteria if the source material is not completely sterile. In vitro-cultured buds were obtained from sugarcane cultivars.

Plantlets and in vitro-cultured buds were separated with approximately 3-5 tillers/buds as necessary, making sure to preserve the leaf base and meristem regions. Cutting off some leaf blades and roots from whole plantlets may increase induction efficiency, but it is not necessary. Plantlets/buds pieces (about 3-6 pieces per plate) were transferred in DBC3 green tissue induction media on Petri dishes to induce callus or green regenerative tissue. DBC3+100 mg/L carbenicillin may be used to control contamination of endogenous bacteria if the source material is not completely sterile.

The tissue is incubated at 26-28° C. under dark or dim light (0-10 μmol m⁻² sec⁻¹) conditions. After 2-4 weeks, callus/green tissues will be induced mostly from the base (crown) of the plantlet. Isolate and transfer only white, compact embryogenic callus or light green tissue to fresh media; a much higher proportion of callus tissue is induced than green tissue. Do not transfer tissue with a “watery” morphology. The tissues were broken into small pieces, about 2-3 mm in diameter. The plate is sealed and incubated at 26-28° C. under dark or dim light conditions for 3-4 weeks until there is enough mass of tissue.

Continue proliferating and maintaining white, compact embryogenic callus or light green tissue every 3 weeks onto fresh DBC3 media as necessary. Tissue that is younger than 2 months old is considered ideal for transformation. The process can be repeated, ideally with fresh plantlets/in vitro-cultured buds, on a regular basis.

### B. Agrobacterium-Mediated Transformation of Sugarcane Callus

### Preparation of Agrobacterium Suspension:

Agrobacterium tumefaciens harboring a binary vector were streaked out from a −80° frozen aliquot onto solid LB medium containing 100 mg/L spectinomycin and cultured at 28° C. in the dark for 2-3 days. A single colony or multiple colonies were picked from the master plate and streaked onto a plate containing 810D medium. The plate was incubated at 28° C. in the dark for 1-2 days. Agrobacterium cells were collected from the solid medium using 5 mL 10 mM MgSO₄ medium (Agrobacterium infection medium) containing 100 μM acetosyringone. The OD of the suspension was adjusted to 0.35-0.40 at 550 nm using the same medium.

### Agrobacterium Infection and Cocultivation:

Good quality callus/green tissues was collected in an empty Petri dish and exposed to air in the hood for about 30 minutes. Add 1 mL Agrobacterium suspension to the Petri dish, break/chop the tissues into small pieces and add additional 1-3 mL Agrobacterium suspension to cover all the chopped tissues.

The Petri dish was placed into a transparent polycarbonate desiccator container, cover the desiccator container and connect to an in-house vacuum system for 20 minutes. After infection, the Agrobacterium suspension was drawn off from the Petri dish and the tissues were transferred onto 2 layers of VWR 415 filter paper (7.5 cm diameter) or miracloth of a new Petri dish with 0.7-1.0 mL liquid DBC3 (M5G) medium added.

The top layer of filter paper or miracloth containing the infected tissues was transferred to a fresh layer of filter paper or miracloth of another new Petri dish. The infected tissues were incubated at 21° C. in the dark for 3 days. The tissues were then transferred to DBC 3 resting medium containing antibiotics (cefotaxime+timentin) without a selective agent and were incubated at 26-28° C. in dark or dim light conditions for 3 days.

### Selection and Plant Regeneration:

Callus was transferred to first round selection DBC3 containing antibiotics (timentin and cefotaxime) and 3 mg/L bialaphos (Meiji Seika, Tokyo, Japan) for bar, pat, or moPAT selection. The callus was subcultured for 3 weeks at 26-28° C. in dark or dim light conditions. Callus was transferred to second round selection DBC6 containing antibiotics and 3-5 mg/L bialaphos. The callus was subcultured for 3 weeks at 26-28° C. in dark or dim light conditions. Tissues were broken as necessary. At the 3rd round selection on DBC6 medium (containing antibiotics and bialaphos), tissues were broken into smaller pieces and exposed to bright light conditions (30-150 μmol m⁻² sec⁻¹) for 2-3 weeks.

Shoot elongated tissues were broken into small pieces and transferred to MSB regeneration/rooting medium containing antibiotics and 3 mg/L bialaphos. Single plantlets were separated and transferred to soil.

### Example 7

### Sugarcane Transformation Using Developmental Genes

A developmental gene binary vector with the BBM/WUS gene cassette was initially compared with a standard vector containing moPAT plus either DsRED or YFP without the BBM/WUS gene cassette for transformation frequency using two Agrobacterium strains, AGL1 and LBA4404, in CP89-2376 and CP01-1372 cultivars. The developmental gene binary vector contains Ubi::LoxP::CFP+Rab17Pro-attb1::Cre+Nos::ZmWUS2+Ubi::ZmBBM-LoxP::YFP+Ubi:: MOPAT. The Lox cassette containing CFP::Cre::WUS::BBM can be excised by Cre recombinase controlled by the Rab17 promoter. Callus tissues of both CP89-2376 and CP01-1372 cultivars were induced and maintained on DBC3 medium. Tissues were infected with Agrobacterium containing the developmental gene binary vector in liquid 10 mM MgSO₄ plus 100 μM acetosyringone and then co-cultivated with liquid DBC3 (M5G) medium plus 100 μM acetosyringone on the filter paper in Petri dishes at 21° C. in the dark. Three days after co-cultivation the tissues were transferred to DBC3 containing 100 mg/L cefotaxime and 150 mg/L timentin for AGL1 and DBC3 containing 100 mg/L carbenicillin for LBA4404, and incubated at 26° C. (±1° C.) in the dark or dim light for 3-7 days. Afterwards, the tissues were transferred to the same media as the previous step plus 3 or 5 mg/L bialaphos. After 2 to 3 weeks, the tissues were transferred to 2nd round selection DBC6 containing antibiotics and 3-5 mg/L bialaphos. After two months from the initiation of the experiment, transformation frequency was calculated by the number of tissues showing CFP expression divided by the number of explants infected by Agrobacterium. Table 6 demonstrated that AGL1 was even more efficient in transformation than LBA4404 in both CP89-2376 and CP01-1372. There was also a genotype difference in transformation frequency; CP89-2376 had much higher transformation frequencies than CP01-1372 using either of the Agrobacterium strains.

AGL1 containing the developmental gene vector was also used to test sugarcane germplasm screening in another set of experiments using 5 different cultivars (CP96-1252, CP01-1372, CP89-2376, CPCL97-2730 and HoCP85-845). Callus tissues of all 5 cultivars tested were induced and maintained on DBC3 medium and tissues were infected with AGL1 containing the developmental gene binary vector. The use of developmental genes dramatically increased transformation frequency in all 5 cultivars tested. Transformation frequencies in the most amenable cultivar, CP89-2376, using a standard binary vector averaged 116.7% (56/48) (Table 6). In contrast, an average transformation frequency in this cultivar from 5 experiments was >2,512.5% (>1,005 events/40 tissues infected) using the developmental gene binary vector. Similar results were obtained from the remaining 4 cultivars, CP96-1252, CP01-1372, CPCL97-2730 and HoCP85-845; transformation frequencies ranged from 62.5% to 187.5% in these 4 cultivars while no transgenic events were obtained using the standard vector without the BBM/WUS gene cassette from these cultivars.

TABLE 6
Transformation Frequency in Sugarcane Using
the BBM/WUS Developmental Gene Cassettes

| Agrobacterium | | | | | | |
|---|---|---|---|---|---|---|
| | Binary | Sugarcane Cultivar | | | | |
| Strain | Vector | CP96-1252 | CP01-1372 | CP89-2376 | CPCL97-2730 | HoCP85-845 |
| AGL1 | DG^{a} | n.t.^{c} | 37.5% | n.t. | n.t. | n.t. |
| | | | (3/8) | | | |
| LBA4404 | DG | n.t. | 0% | n.t. | n.t. | n.t. |
| | | | (0/8) | | | |
| AGL1 | DG | n.t. | >1,250.0% | >6,250.0% | n.t. | n.t. |
| | | | (>100/8) | (>500/8) | | |
| LBA4404 | DG | n.t. | 12.5% | >1,500% | n.t. | n.t. |
| | | | (1/8) | (>120/8) | | |
| AGL1 | DG | n.t. | n.t. | 687.5% | n.t. | n.t. |
| | | | | (>55/8) | | |
| AGL1 | DG | n.t. | n.t. | >2,500% | 175.0% | n.t. |
| | | | | (>200/8) | (14/8) | |
| AGL1 | DG | 150.0% | 62.5% | >625.0% | 62.5% | n.t. |
| | | (12/8) | (5/8) | (>50/8) | (6/8) | |
| AGL1 | DG | n.t. | n.t. | >2,500% | n.t. | 187.5% |
| | | | | (>200/8) | | (15/8) |
| AGL1 | Std^{b} | 0% | 0% | 116.7% | 0% | 0% |
| | | (0/8) | (0/8) | (56/48) | (0/8) | (0/8) |

Each transformation treatment had 8 pieces of callus tissues 0.4-0.5 cm in size.
DG^{a} developmental gene vector with BBM/WUS gene cassette
Std^{b} standard vector without BBM/WUS gene cassette
n.t.^{c} not tested

Transgenic callus tissues were desiccated on dry filter papers for three days to induce excision of the Lox cassette containing CFP::Cre::WUS::BBM by Cre recombinase driven by the Rab17 promoter. Excision was monitored by observing YFP expression on desiccated transgenic callus events by the presence of the UBI:loxP:YFP junction formed as a result of excision. Cre excision occurred at 83 of 87 transgenic events (95.4%) (Table 7). Plants from some transgenic events after excision wereregenerated on MSB plus 1 mg/L bialaphos and antibiotics.

TABLE 7
Excision Efficiency of the BBB/WUS Gene Cassette in
Transgenic Sugarcane Events by Desiccation

| Sugarcane | Agrobacterium | Binary | Excision |
|---|---|---|---|
| Cultivar | Strain | Vector | Efficiency (%) |
| CP89-2376 | AGL1 | DG^{a} | 93% (40/43) |
| CP89-2376 | LBA4404 | DG | 100% (25/25) |
| CP01-1372 | AGL1 | DG | 100% (13/13) |
| CP01-1372 | LBA4404 | DG | 0% (0/1) |
| CP89-2376 | AGL1 | DG | 100% (5/5) |

| | |
|---|---|
| Average | 95.4% (83/87) |

DG^{a}: developmental gene vector with BBM/WUS gene cassette

### Example 8

### Visual Marker Selection vs Bialaphos Selection for Sugarcane Transformation

Visual marker selection and moPAT selection were compared for transformation frequency in CP89-2376. Two sets of tissues were infected with Agrobacterium AGL1 containing pDsREDPAT in liquid 10 mM MgSO₄ plus 100 μM acetosyringone and broken/chopped into small pieces. The tissues were then co-cultivated with liquid DBC3(M5G) medium plus 100 μM acetosyringone on the filter paper at 21° C. in the dark. Three days after co-cultivation the tissues were transferred to DBC3+100 mg/L cefotaxime+150 mg/L timentin, and incubated at 26° C. (±1° C.) in the dark or dim light for 3-7 days. One set of the tissues was then transferred to DBC3+100 mg/L cefotaxime+150 mg/L timentin without bialaphos and the other set to the DBC3+100 mg/L cefotaxime+150 mg/L timentin medium plus 3 mg/L bialaphos. After 2 to 3 weeks, the tissues for bialaphos selection were transferred to 2nd round selection on DBC6 medium containing antibiotics and 3-5 mg/L bialaphos. In contrast tissues for visual marker selection tissues were transferred to DBC6 containing antibiotics and no bialaphos. After about 1.5 months DsRED sectors were selected by visual marker selection using a fluorescence microscope and transferred to DBC6 containing antibiotics and no bialaphos for proliferation.

After the proliferation step transgenic tissue events were cultured on MSB regeneration medium with or without 1 or 3 mg/L bialaphos using the regeneration conditions as described in Example 6. Transformation frequency was calculated by the number of explants producing transgenic plants divided by the number of explants infected by Agrobacterium. Table 8 shows that transformation frequency was almost same or slightly higher using visual marker selection compared with bialaphos selection.

TABLE 8
Transformation Frequency in Sugarcane (cv. CP89-2376) Using
Visual Marker Selection and Bialaphos Selection

| | Visual marker selection | Bialaphos selection |
|---|---|---|
| Expt1 | 137.5% (11/8) | 100.0% (8/8) |
| Expt 2 | 125.0% (10/8) | 112.5% (9/8) |
| Average | 131.3% (21/16) | 106.3% (17/16) |

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of regenerating a plant, the method comprising the steps of:
(a) culturing an in vitro-cultured plantlet or in vitro-cultured bud in the presence of a medium to induce callus or green regenerative tissue formation;
(b) culturing the callus or green regenerative tissue in the presence of a medium to regenerate the plant.

2. The method of claim 1, wherein the plantlet is an in vitro-cultured whole plantlet.

3. The method of claim 1, wherein roots of the plantlet are removed prior to culturing step (a).

4. The method of claim 1, wherein the monocotyledonous plantlet or in vitro cultured bud is sugarcane.

5. The method of claim 1, further comprising introducing a nucleic acid into a cell of the callus or green regenerative tissue to produce a transformed plant cell.

6. The method of claim 1, wherein the medium of (a) comprises at least one cytokinin and at least one auxin, and further comprises at least one of maltose, sucrose, copper, thiamine-HCl, myo-inositol, N—Z-amine-A (casein hydrolysate), and proline.

7. The method of claim 6, wherein the at least one cytokinin are BAP and kinetin and the at least one auxin are 2,4-D and dicamba.

8. The method of claim 1, wherein medium of (a) the cytokinin is benzylaminopurine (BAP) and the auxin is 2,4-dichlorophenoxyacetic acid (2,4-D).

9. The method of claim 8, wherein the medium of (a) comprises about 0.01 to about 5 milligrams/L BAP, about 0.1 to about 5 milligrams/L 2,4-D, about 0.1 to about 20 μM copper, about 0.25 grams/L myo-inositol, about 1 gram/L N—Z-amine-A (casein hydrolysate), about 0.7 grams/L proline, and about 30 gram/L maltose or sucrose.

10. A method for producing a transformed plant, comprising the steps of:
(a) culturing a in vitro-cultured plantlet or in vitro-cultured bud in the presence of an medium to induce callus or green regenerative tissue formation;
(b) introducing a nucleic acid into a cell of the callus or green regenerative tissue to produce a transformed plant cell,
(c) culturing the transformed plant cell in the presence of a medium, thereby promoting proliferation and formation of a transformed structure that is competent to regenerate; and
(d) culturing the transformed structure in the presence of a medium to produce the transformed plant.

11. The method of claim 10, wherein the introducing step comprises Agrobacterium-mediated transformation.

12. The method of claim 10, wherein the medium of (a) comprises at least one cytokinin, at least one auxin, and further comprises at least one of maltose, sucrose, copper, thiamine-HCl, myo-inositol, N—Z-amine-A (casein hydrolysate), and proline.

13. The method of claim 12, wherein the at least one cytokinin are BAP and kinetin and the at least one auxin are 2,4-D and dicamba.

14. The method of claim 12, wherein the medium of (a) the cytokinin is benzylaminopurine (BAP) and the auxin is 2,4-dichlorophenoxyacetic acid (2,4-D).

15. The method of claim 14, wherein the medium of (a) comprises about 0.01 to about 5 milligrams/L BAP, about 0.1 to about 5 milligrams/L 2,4-D, about 0.1 to about 20 μM copper, about 0.25 grams/L myo-inositol, about 1 gram/Liter N—Z-amine-A (casein hydrolysate), about 0.7 grams/L proline, and about 30 gram/L maltose or sucrose.

16. The method of claim 10, further comprising selecting for the transformed plant cell by incubating the callus or green regenerative tissue in the presence of a medium comprising a selective agent.

17. The method of claim 10, wherein the plant, bud culture, or plantlet is sugarcane.

18. A method for producing a transformed plant, comprising the steps of:
(a) culturing a whole plantlet or in vitro-cultured bud in the presence of a medium to induce callus or green regenerative tissue formation;
(b) contacting the callus or green regenerative tissue with an Agrobacterium comprising a vector which comprises a polynucleotide, wherein the polynucleotide comprises an expression cassette comprising a gene which confers resistance to a selection agent;
(c) co-cultivating the tissue with the Agrobacterium;
(d) selecting regenerable cells comprising the polynucleotide; and
(e) culturing the regenerable cells in the presence of a regeneration medium to produce the transformed plant.

19. The method of claim 18, further comprising culturing the tissue of step (c) in a medium comprising a compound capable of inhibiting the growth of Agrobacterium and the selection agent.

20. The method of claim 18, wherein the medium of (a) comprising at least one cytokinin and at least one auxin.

21. The method of claim 20, wherein the at least one cytokinin are BAP and kinetin and the at least one auxin are 2,4-D and dicamba.

22. The method of claim 20, wherein the medium of (a) wherein the cytokinin is benzylaminopurine (BAP) and the the auxin is 2,4-dichlorophenoxyacetic acid (2,4-D), and further comprises at least one of maltose, sucrose, copper, thiamine-HCl, myo-inositol, N—Z-amine-A (casein hydrolysate), and proline.

23. The method of claim 22, wherein the medium of (a) comprises about 0.01 to about 5 milligrams/Liter BAP, about 0.1 to about 5 milligrams/Liter 2,4-D, about 1.0 to about 20 μM copper, about 0.25 grams/Liter myo-inositol, about 1 gram/Liter N—Z-amine-A (casein hydrolysate), about 0.7 grams/Liter proline, and about 30 grams/L maltose or sucrose.

24. The method of claim 18, wherein the medium of (c) comprises about 4.43 grams/L MS salts and vitamins, 20 grams/L sucrose, 1 gram/L myo-inositol and 3.5 grams/L Phytagel.

25. The method of claim 18, wherein the plant, plant bud, or plantlet is sugarcane.

26. The method of claim 18, wherein the polynucleotide further comprises a developmental gene cassette.

27. The method of claim 26, wherein the developmental gene cassette encodes at least one cell proliferation transcription factor selected from the group consisting of either of babyboom (BBM) or Wuschel (WUS), or both.


SEQUENCE LISTING (12 sequences)

SEQ ID NO: 1 - DNA, 1755 bp, Arabidopsis thaliana
atgaactcgatgaataactggttaggcttctctctctctcctcatgatcaaaatcatcaccgtacggatgttgactcctccaccaccagaaccgccgtagatgttgccggagggtactgttttgatctggccgctccctccgatgaatcttctgccgttcaaacatcttttctttctcctttcggtgtcaccctcgaagctttcaccagagacaataatagtcactcccgagattgggacatcaatggtggtgcatgcaatacattaaccaataacgaacaaaatggaccaaagcttgagaatttcctcggccgcaccaccacgatttacaataccaacgagaccgttgtagatggaaatggcgattgtggaggaggagacggtggtggtggcggctcactaggcctttcgatgataaaaacatggctgagtaatcattcggttgctaatgctaatcatcaagacaatggtaacggtgcacgaggcttgtccctctctatgaattcatctactagtgatagcaacaactacaacaacaatgatgatgtcgtccaagagaagactattgttgatgtcgtagaaactacaccgaagaaaactattgagagttttggacaaaggacgtctatataccgcggtgttacaaggcatcggtggacaggtagatacgaggcacatttatgggacaatagttgcaaaagagaaggccagactcgcaaaggaagacaagtttatctgggaggttatgacaaagaagaaaaagcagctagggcttacgatttagccgcactaaagtattggggacccaccactactactaacttccccttgagtgaatatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaaagacctctacttgggaactttcggcacacaggaagaggctgctgaggcttatgacattgcagccattaaattcagaggattaagcgcagtgactaacttcgacatgaacagatacaatgttaaagcaatcctcgagagcccgagtctacctattggtagttctgcgaaacgtctcaaggacgttaacaatccggttccagctatgatgattagtaataacgtttcagagagtgcaaataatgttagcggttggcaaaacactgcgtttcagcatcatcagggaatggatttgagcttattgcagcaacagcaggagaggtacgttggttattacaatggaggaaacttgtctaccgagagtactagggtttgtttcaaacaagaggaggaacaacaacacttcttgagaaactcgccgagtcacatgactaatgttgatcatcatagctcgacctctgatgattctgttaccgtttgtggaaatgttgttagttatggtggttatcaaggattcgcaatccctgttggaacatcggttaattacgatccctttactgctgctgagattgcttacaacgcaagaaatcattattactatgctcagcatcagcaacaacagcagattcagcagtcgccgggaggagattttccggtggcgatttcgaataaccatagctctaacatgtactttcacggggaaggtggtggagaaggggctccaacgttttcagtttggaacgacacttag

SEQ ID NO: 2 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNTLTNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGPTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 3 - DNA, 2130 bp, Zea mays
atggccactgtgaacaactggctcgctttctccctctccccgcaggagctgccgccctcccagacgacggactccacgctcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaagtccaactgcaacttgatacccagcactagcagcacagtttgctacgcgagctcagctgctagcaccggctaccatcaccagctgtaccagcccaccagctccgcgctccacttcgcggactccgtcatggtggcctcctcggccggtgtccacgacggcggttccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaaggacaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgtctcaaggaggccgaggccgcagcgtccgcgcagcaccaccacgccggcgtggtgagctacgacgtcggccgcatcgcctcgcagctcggcgacggcggagccctagcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacttgaacctgggcgcggccggcgcgcacgactttttctcggcagggcagcaggccgccgccgcagctgcgatgcacggcctggctagcatcgacagtgcgtcgctcgagcacagcaccggctccaactccgtcgtctacaacggcggggtcggcgatagcaacggcgccagcgccgttggcagcggcggtggctacatgatgccgatgagcgctgccggagcaaccactacatcggcaatggtgagccacgagcagatgcatgcacgggcctacgacgaagccaagcaggctgctcagatggggtacgagagctacctggtgaacgcggagaacaatggtggcggaaggatgtctgcatgggggaccgtcgtctctgcagccgcggcggcagcagcaagcagcaacgacaacattgccgccgacgtcggccatggcggcgcgcagctcttcagtgtctggaacgacacttaa

SEQ ID NO: 4 - protein, 709 aa, Zea mays
MATVNNWLAFSLSPQELPPSQTTDSTLISAATADHVSGDVCFNIPQDWSMRGSELSALVAEPKLEDFLGGISFSEQHHKSNCNLIPSTSSTVCYASSAASTGYHHQLYQPTSSALHFADSVMVASSAGVHDGGSMLSAAAANGVAGAASANGGGIGLSMIKNWLRSQPAPMQPRAAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAATTGLYHPYAQQPMRGGGWCKQEQDHAVIAAAHSLQDLHHLNLGAAGAHDFFSAGQQAAAAAAMHGLASIDSASLEHSTGSNSVVYNGGVGDSNGASAVGSGGGYMMPMSAAGATTTSAMVSHEQMHARAYDEAKQAAQMGYESYLVNAENNGGGRMSAWGTVVSAAAAAAASSNDNIAADVGHGGAQLFSVWNDT

SEQ ID NO: 5 - DNA, 975 bp, Zea mays
atggagacgccacagcagcaatccgccgccgccgccgccgccgccgcccacgggcaggacgacggcgggtcgccgccgatgtcgccggcctccgccgcggcggcggcgctggcgaacgcgcggtggaacccgaccaaggagcaggtggccgtgctggaggggctgtacgagcacggcctgcgcacccccagcgcggagcagatacagcagatcacgggcaggctgcgggagcacggcgccatcgagggcaagaacgtcttctactggttccagaaccacaaggcccgccagcgccagaggcagaagcaggacagcttcgcctacttcagcaggctcctccgccggcccccgccgctgcccgtgctctccatgccccccgcgccaccgtaccatcacgcccgcgtcccggcgccgcccgcgataccgatgccgatggcgccgccgccgcccgctgcatgcaacgacaacggcggcgcgcgtgtgatctacaggaacccattctacgtggctgcgccgcaggcgccccctgcaaatgccgcctactactacccacagccacagcagcagcagcagcagcaggtgacagtcatgtaccagtacccgagaatggaggtagccggccaggacaagatgatgaccagggccgcggcgcaccagcagcagcagcacaacggcgccgggcaacaaccgggacgcgccggccaccccagccgcgagacgctccagctgttcccgctccagcccaccttcgtgctgcggcacgacaaggggcgcgccgccaacggcagtaataacgactccctgacgtcgacgtcgacggcgactgcgacagcgacagcgacagcgacagcgtccgcttccatctccgaggactcggatggcctggagagcggcagctccggcaagggcgtcgaggaggcgcccgcgctgccgttctatgacttcttcgggctccagtcctccggaggccgctga

SEQ ID NO: 6 - protein, 324 aa, Zea mays
METPQQQSAAAAAAAAHGQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHARVPAPPAIPMPMAPPPPAACNDNGGARVIYRNPFYVAAPQAPPANAAYYYPQPQQQQQQQVTVMYQYPRMEVAGQDKMMTRAAAHQQQQHNGAGQQPGRAGHPSRETLQLFPLQPTFVLRHDKGRAANGSNNDSLTSTSTATATATATATASASISEDSDGLESGSSGKGVEEAPALPFYDFFGLQSSGGR

SEQ ID NO: 7 - DNA, 909 bp, Zea mays
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtga

SEQ ID NO: 8 - protein, 302 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 9 - DNA, 978 bp, Zea mays
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccttcaggcgcggcgcctccctcgcccaccctcggcttctacgccgccggcaatggcggcggatcggctgggctgctggacacgagttccgactggggcagcagcggcgctgctatggccaccgagacatgcttcctgcaggactacatgggcgtgacggacacgggcagctcgtcgcagtggccatgcttctcgtcgtcggacacgataatggcggcggcggcggccgcggcgcgggtggcgacgacgcgggcgcccgagacactccctctcttcccgacctgcggcgacgacgacgacgacgacagccagcccccgccgcggccgcggcacgcagtcccagtcccggcaggcgagaccatccgcggcggcggcggcagcagcagcagctacttgccgttctggggtgccggtgccgcgtccacaactgccggcgccacttcttccgttgcgatccagcagcaacaccagctgcaggagcagtacagcttttacagcaacagcacccagctggccggcaccggcagccaagacgtatcggcttcagcggccgccctggagctgagcctcagctcatggtgctccccttaccctgctgcagggagcatgtga

SEQ ID NO: 10 - protein, 325 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPSGAAPPSPTLGFYAAGNGGGSAGLLDTSSDWGSSGAAMATETCFLQDYMGVTDTGSSSQWPCFSSSDTIMAAAAAAARVATTRAPETLPLFPTCGDDDDDDSQPPPRPRHAVPVPAGETIRGGGGSSSSYLPFWGAGAASTTAGATSSVAIQQQHQLQEQYSFYSNSTQLAGTGSQDVSASAAALELSLSSWCSPYPAAGSM

SEQ ID NO: 11 - DNA, 663 bp, Zea mays
atggaggcgctgagcgggcgggtaggcgtcaagtgcgggcggtggaaccctacggcggagcaggtgaaggtcctgacggagctcttccgcgcggggctgcggacgcccagcacggagcagatccagcgcatctccacccacctcagcgccttcggcaaggtggagagcaagaacgtcttctactggttccagaaccacaaggcccgcgagcgccaccaccacaagaagcgccgccgcggcgcgtcgtcgtcctcccccgacagcggcagcggcaggggaagcaacaacgaggaagacggccgtggtgccgcctcgcagtcgcacgacgccgacgccgacgccgacctcgtgctgcaaccgccagagagcaagcgggaggccagaagctatggccaccatcaccggctcgtgacatgctacgtcagggacgtggtggagcagcaggaggcgtcgccgtcgtgggagcggccgacgagggaggtggagacgctagagctcttccccctcaagtcgtacggcgacctcgaggcggcggagaaggtccggtcgtacgtcagaggcatcgccgccaccagcgagcagtgcagggagttgtccttcttcgacgtctccgccggccgggatccgccgctcgagctcaggctctgcagcttcggtccctag

SEQ ID NO: 12 - protein, 220 aa, Zea mays
MEALSGRVGVKCGRWNPTAEQVKVLTELFRAGLRTPSTEQIQRISTHLSAFGKVESKNVFYWFQNHKARERHHHKKRRRGASSSSPDSGSGRGSNNEEDGRGAASQSHDADADADLVLQPPESKREARSYGHHHRLVTCYVRDVVEQQEASPSWERPTREVETLELFPLKSYGDLEAAEKVRSYVRGIAATSEQCRELSFFDVSAGRDPPLELRLCSFGP